(19) United States
(12) Patent Application Publication
(10) Pub. No.: US 2011/0190216 A1
(43) Pub. Date: Aug. 4, 2011

(54) MATERIALS AND METHODS FOR TREATMENT OF SPINAL MUSCULAR ATROPHY AND TAXANE-INDUCED PERIPHERAL NEUROPATHY (TIPN)

(75) Inventor: Kate Calvin, Monticello, FL (US)

(73) Assignee: Florida State University Research Foundation, Tallahassee, FL (US)

(21) Appl. No.: 13/056,499

(22) PCT Filed: Jul. 29, 2009
(86) PCT No.: PCT/US2009/052150

§ 371 Date: Apr. 25, 2011

Related U.S. Application Data
(60) Provisional application No. 61/084,556, filed on Jul. 29, 2008.

(51) Int. Cl. A61K 38/17; C07K 19/00; A61K 31/7088; C12N 5/0793; A61P 25/28; A61P 25/00

(52) U.S. Cl. 514/17.7; 530/350; 514/44 R; 435/375

(57) ABSTRACT

The present invention concerns materials and methods for treating, inhibiting the progression or, and/or preventing a disorder associated with and/or characterized by neuronal degeneration, such as SMA or TIPN, in a person or animal. One aspect of the invention pertains to a fusion protein comprising: i) an SMN polypeptide portion, or a fragment or variant thereof having SMN biological activity, and ii) a non-toxic BoTN portion, or a fragment or variant thereof capable of providing for receptor-mediated endocytosis in a cell, such as a neuron. In one embodiment, the SMN protein is a human SMN1 protein. In one embodiment, the BoTN portion comprises the BoTN heavy chain, or a fragment or variant thereof capable of providing for receptor-mediated endocytosis in a cell. The non-toxic BoTN portion can optionally comprise a modified and/or hybrid polypeptide that comprises amino acid sequences or polypeptides from non-BoTN proteins or polypeptides and optionally BoTN polypeptides. For example, in one embodiment, a non-toxic BoTN portion of the invention comprises a non-toxic portion of a diphtheria toxin and/or tetanus toxin.

### CROSS-REFERENCE TO RELATED APPLICATION

The present application claims the benefit of U.S. Provisional Application Ser. No. 61/084,556, filed Jul. 29, 2008, which is hereby incorporated by reference herein in its entirety, including any figures, tables, nucleic acid sequences, amino acid sequences, and drawings.

### BACKGROUND OF THE INVENTION

Spinal muscular atrophy (SMA) and taxane-induced peripheral neuropathy (TIPN) are two very different neuronal disorders with some distinctly overlapping features. SMA is caused by a genetic mutation that knocks down the expression of the essential protein “survival motor neuron,” or SMN, which has been shown to be vital for axon terminal maintenance in neurons. SMN gene therapy strategies have shown promise but require local injection and pharmaceutical formulations that promote SMN gene expression are also being studied. However, a multi-pronged strategy for ameliorating SMA symptoms is needed to maximize treatment efficacy. Taxane-induced peripheral neuropathy is a painful and sometimes debilitating side-effect of anti-cancer chemotherapeutics that function as microtubule stabilizing agents (MTSAs). TIPN affects primarily sensory neurons but motor neuronal symptoms are also reported. In neurons, microtubule networks are very long and serve as the transport “highways” for essential cargo to move between the cell body and axon terminal. MTSAs are believed to block the transport of molecules needed for axonal maintenance, though the precise mechanism underlying TIPN is not known. Many treatments for TIPN have been studied, a few of which are promising, though none sheds significant light on the underlying mechanism. Despite differences in relative effects, both SMA and TIPN produce motor and sensory axonal degeneration.

Spinal muscular atrophy (SMA) is a motor neuron disease and is the leading genetic cause of infant mortality. It is a fairly common autosomal recessive disorder, with as many as one in forty people carrying the genetic mutation and approximately one in every 6000 babies being affected by the disease. The critical gene encodes the survival motor neuron (SMN) protein. While SMN is essential in all cells, motor neurons emanating from the spinal cord anterior horn are especially affected when the functional protein is absent. In extreme cases of SMA, the loss of motor function results in impaired swallowing, breathing and ability to control head movements. Infants and toddlers so afflicted often develop secondary respiratory infections and do not survive past the age of two (Markowitz et al., 2004). SMN is transported along neuronal microtubule “highways” (Gunawardena and Goldstein, 2004; Zhai and Bellen, 2004) via fast axonal transport, both anterogradely and retrogradely (Zhang et al., 2003) and it has been found to be essential in motor and sensory neurons for growth cone development, neurite out growth, axon outgrowth and maintenance of axon termini (Jablonka et al., 2006; McWhorter et al., 2003; Carrel et al., 2006). The specific absence of SMN has been shown to result in motor and sensory nerve axonal degeneration (Balabanian et al., 2007; Cifuentes-Diaz et al., 2002; Omran et al., 1998; Rudnik-Schoneborn et al., 2003). Sensory nerves are affected in severe SMA cases as well, though not as significantly (Jablonka et al., 2006). Symptoms include atrophic axons and, in some cases, altogether unexcitable neurons (Jablonka et al., 2006; Omran et al., 1998; Rudnik-Schonebom et al., 2003). SMN's name is somewhat deceiving because this protein is essential to all cells of the body and it has been called the “Master Assembler” (Terns and Terns, 2001) because it plays an integral role in recruiting together components of several macromolecular complexes including the spliceosome. SMN interacts with a multitude of proteins and ribonucleic acids (RNAs) and its multifaceted role in various cell types is still being elucidated (Carrel et al., 2006; Zhang et al., 2008; Zou et al., 2007; Vitte et al., 2004; Shanmugarajan et al., 2007).

Two genes encode human SMN protein: smn1 and smn2. The differences between them are their location on chromosome 5q13 and their activity: smn1 is telomeric and encodes functional protein, while smn2 is centromeric and produces inactive protein (McWhorter et al., 2003). The human smn1 gene shares 81% sequence identity with the single copy of mouse smn (Viollet et al., 1997). Studies have been done using transgenic mice that express human smn2 (Hua et al., 2008; Schmalbruch and Haase, 2001) and smn1 (Monani et al., 2003), and human smn1 in rat (Vyas et al., 2002), but specific structural differences between mouse SMN and human SMN1 proteins, aligned in FIG. 1, have not been identified. Attempts have been made to deliver human SMN protein directly to rat nerve terminals via endocytosis of a recombinant tetanus toxin fragment; however, these efforts were not successful because of a problem with the human SMN moiety (Francis et al., 2004).

Taxane-based medications such as paclitaxel and docetaxel are very successful anti-cancer treatments. They are microtubule stabilizing agents that bind directly to microtubule polymers and independently polymerize tubulin, a protein component of microtubules (Horwitz, 1994; Ganasia-Leymarie et al., 2003). This binding activity prevents the normal dynamic assembly and disassembly of microtubules, inhibits cell division and induces cell death (Horwitz, 1994; Ganasia-Leymarie et al., 2003). These chemotherapeutic treatments are effective against cancer cells but they also damage other cell types, including sensory neurons and also motor neurons to a lesser extent. Painful side effects associated with peripheral sensory neuropathy have been documented (Lee and Swain, 2006; Markman, 2003; Argyriou et al., 2008) with some symptoms being quite severe and persisting for years (Lee and Swain, 2006; Peters et al., 2007). The neuronal damage induced by paclitaxel has several features consistent with axotomy and impaired fast axonal transport to distal termini (Viollet et al., 1997; Schmalbruch and Haase, 2001). Reduced axonal transport of proteins and/or RNAs essential to axonal maintenance has been shown to result in both endogenous and exogenous neuronal disorders (Zhai and Bellen, 2004; Argyriou et al., 2008; Rao and Nixon, 2003). Consequently, impaired axonal transport is generally believed to play a role in taxane-induced peripheral neuropathy (TIPN), though the precise mechanisms underlying this disorder remain unclear (Lee and Swain, 2006; Argyriou et al., 2008; Jimenez-Andrade, 2006). Other possible mechanisms include induction of apoptotic signaling cascades (Ganasia-Leymarie et al., 2003) and damage to neuronal support cells (Jimenez-Andrade, 2006; Mielke et al., 2006). Many treatments for TIPN are being studied, though none provides specific information on how the disorder arises at the molecular level.

The general commonalities between SMA and TIPN include abnormal accumulation of neurofilaments (Cifuentes-Diaz et al., 2002; Jimenez-Andrade, 2006), aberrant growth cone development alongside damage to sensory and motor neurons (Jablonka et al., 2006; Lee and Swain, 2006), with shared sensory neuronal damage observed largely in the sural nerve (Omran et al., 1998; Rudnik-Sehoneborn et al., 2003; Sahenk et al., 1994; Fazio et al., 1999). A potential link between SMA and TIPN is SMN protein. It is transported to axon termini via fast axonal transport, which is impaired by TIPN. The end result in both cases is a lack of SMN protein in axon termini.

Botulinum neurotoxin (BoNT) is the causative agent of botulism in humans and other mammals including mice. Before it can enter the bloodstream, BoNT must travel through gut, pulmonary, and other epithelial membranes. From the bloodstream, BoTN then typically binds to the presynaptic membrane of neuromuscular junctions and enters the neuronal cytosol via receptor-mediated endocytosis. In the neuron, BoNT characteristically blocks release of acetylcholine at the neuromuscular junction, causing flaccid paralysis of the muscle (U.S. Published Application No. 2004/0013687).

BoNT is produced by the bacterium Clostridium botulinum and currently has seven immunologically distinct forms: A, B, C, D, E, F, and G. All serotypes are produced in association with two kinds of auxiliary proteins: hemagglutinins (“HA”) and a single, nontoxin, non-hemagglutinin protein (“NTNH”). These proteins are believed to stabilize the toxin molecule and protect it from denaturation after ingestion (U.S. Published Application No. 2005/0143289). The toxin molecule contains a light chain and a heavy chain. The heavy chain is the non-toxic binding agent of the molecule. It is responsible for interacting with elements at the nerve terminal to induce endocytosis and, rather than the toxic light chain, it elicits the primary immune responses in vivo (Simpson et al., 1999).

BoTN serotype B specifically binds with high affinity to two presynaptic cell membrane constituents, synaptotagmin (isoforms I and II) and polysialoganglioside GT1b (Zhai and Bellen, 2004; Chai et al., 2006; Lalli et al., 2003; Rummel et al., 2007; Baldwin et al., 2007). Synaptotagmin and GT1b are present in both motor and sensory axon termini (Gong et al., 2002; Li et al., 1994; Meng et al., 2007). Though BoTN characteristically produces muscle paralysis, there are also cases reported where various BoTN serotypes, including type B, have produced a variety of sensory symptoms such as localized numbness (Goode and Shearn, 1982; Sonnabend et al., 1987), partial numbness to one side of the body (Kuruoglu et al., 1996; Martinez-Castrillo et al., 1991), double vision (Kuruoglu et al., 1996; Martinez-Castrillo et al., 1991) and symptoms consistent with mononeuritis multiplex (Goode and Shearn, 1982) and Guillain-Barré syndrome (Sonnabend et al., 1987). Therefore, BoTN serotype B can affect motor and sensory neurons, although higher levels of the toxin are required for sensory effects.

Given that SMN is essential for sensory and motor axon terminal maintenance, there is a need in the art for the delivery of SMN protein directly to nerve terminals to reduce the neuronal degeneration seen in cases of SMA and TIPN.

### BRIEF SUMMARY OF THE INVENTION

The present invention concerns materials and methods for treating or preventing SMA and TIPN in a person or animal. In one embodiment, the present invention concerns a medication that uses botulinum toxin receptor-mediated endocytosis as a tool to deliver SMN protein directly to axon terminals in order to ameliorate the symptoms of SMA and/or TIPN. In the methods, a therapeutically effective amount of a fusion protein or a composition of the invention is administered to a person or animal in need of treatment. In one embodiment, a compound or composition of the invention comprises a fusion protein comprising an SMN protein portion, or a fragment or variant thereof having SMN biological activity, and a non-toxic BoTN heavy chain portion, or a fragment or variant thereof capable of providing for receptor-mediated endocytosis.

The subject invention can also provide specific information regarding how reduced axonal transport and reduced SMN levels in axonal termini contribute to the onset of TIPN.

### BRIEF DESCRIPTION OF THE DRAWINGS

FIG. 1 shows the alignment of human SMN1 (SEQ ID NO:1) and mouse SMN (SEQ ID NO:2), Solid lines between rows indicate exact sequence matches. The proteins share 81% sequence identity.

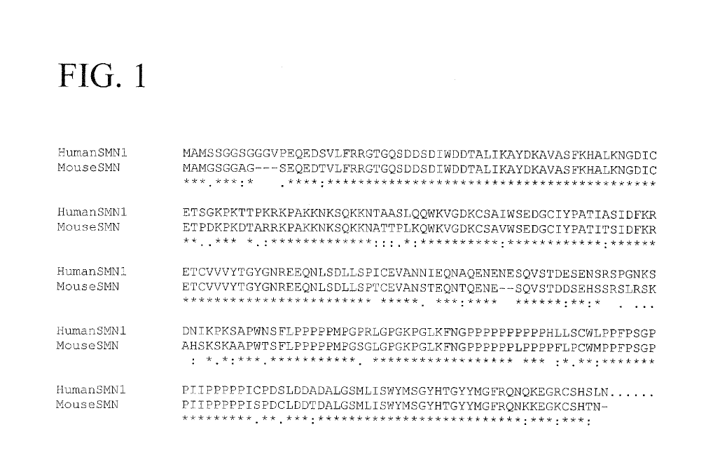

FIG. 2 shows the alignment of the heavy chains from BoTN A (SEQ ID NO:3) and BoTN B (SEQ ID NO:4), which share 42% sequence identity. In the BoTN A rows, the sequence is emphasized in a manner based upon U.S. Published Application No. 2004/0013687. Bold letters indicate the 88 kDa HC fragment, underlining (all types) indicates the 60 kDa HC fragment, double underlining indicate the 50 kDa HC fragment, and italicized letters indicate residues removed in the 48 kDa HC fragment.

FIG. 3 shows schematic drawings of DNA constructs for expression of the SMN-BoTN_B(HC) fusion protein, the heavy chain alone and the fusion protein truncation mutants that can be used according to the present invention. An interchain segment can be used for all the fusion proteins.

### BRIEF DESCRIPTION OF THE SEQUENCES

SEQ ID NO:1 is an amino acid sequence of human SMN1 protein (Accession No. AAH15308).

SEQ ID NO:2 is an amino acid sequence of mouse SMN protein (Accession No. CAA73356).

SEQ ID NO:3 is an amino acid sequence of a botulinum neurotoxin A heavy chain polypeptide.

SEQ ID NO:4 is an amino acid sequence of a botulinum neurotoxin B heavy chain polypeptide.

SEQ ID NO:5 is a nucleotide sequence of a mouse SMN gene (Accession No. NM_{—}011420).

SEQ ID NO:6 is an amino acid sequence of a botulinum neurotoxin B heavy and light chain (Accession No. P10844).

SEQ ID NO:7 is an amino acid sequence of a tetanus toxin heavy chain (amino acids 458-1315 of Accession No. P04958.2).

SEQ ID NO:8 is a nucleotide sequence of a human SMN1 gene (Accession No. BC015308).

SEQ ID NO:9 is amino acids 300-324 (RGRGRGGFDRGGMSRG-GRGGGRGGM) of Ewings sarcoma protein (Sigma).

SEQ ID NO:10 is an amino acid sequence of a peptide tag that can be used according to the present invention.

SEQ ID NO:11 is an amino acid sequence of an interchain amino acid segment that can be used according to the present invention.

SEQ ID NO:12 is an amino acid sequence of an interchain amino acid segment that can be used according to the present invention.

SEQ ID NO:13 is an amino acid sequence of a fusion protein of the present invention.

SEQ ID NO:14 is an amino acid sequence of a fusion protein of the present invention comprising an interchain amino acid sequence of SEQ ID NO:11.

SEQ ID NO:15 is an amino acid sequence of a fusion protein of the present invention comprising an interchain amino acid sequence of SEQ ID NO:12.

SEQ ID NO:16 is an amino acid sequence of an interchain amino acid segment that can be used according to the present invention.

SEQ ID NO:17 is an amino acid sequence of an interchain amino acid segment that can be used according to the present invention.

### DETAILED DESCRIPTION OF THE INVENTION

The present invention concerns compounds, compositions, and methods for treating, inhibiting the progression or, and/or preventing a disorder associated with and/or characterized by neuronal degeneration, such as SMA or TIPN, in a person or animal. Compounds of the invention comprise a fusion protein that includes a portion having SMN biological activity and a portion capable of providing for receptor-mediated endocytosis into a cell. In one embodiment, a compound of the invention comprises a fusion protein that includes: i) an SMN protein portion, or a fragment or variant thereof having SMN biological activity, and ii) a non-toxic botulinum neurotoxin (BoTN) portion, or a fragment or variant thereof capable of providing for receptor-mediated endocytosis in a cell, such as a neuron. In one embodiment, a fusion protein of the invention comprises a binding domain or moiety that binds to a neuronal cell. In a specific embodiment, the binding domain or moiety binds specifically to a neuronal cell. A fusion protein of the invention also comprises a cell membrane translocation domain or moiety that allows the protein to pass through the cell membrane and into the cell.

In one embodiment, the SMN protein of a fusion protein of the invention is a mammalian SMN protein. In one embodiment, the SMN protein is a human SMN1 protein, or a fragment or variant thereof having SMN biological activity. In a specific embodiment, the human SMN1 protein comprises the amino acid sequence shown in SEQ ID NO:1, or a fragment or variant thereof having SMN biological activity. In a more specific embodiment, the fusion protein comprises the amino acid sequence shown in any of SEQ ID NO:13, SEQ ID NO:14, or SEQ ID NO:15.

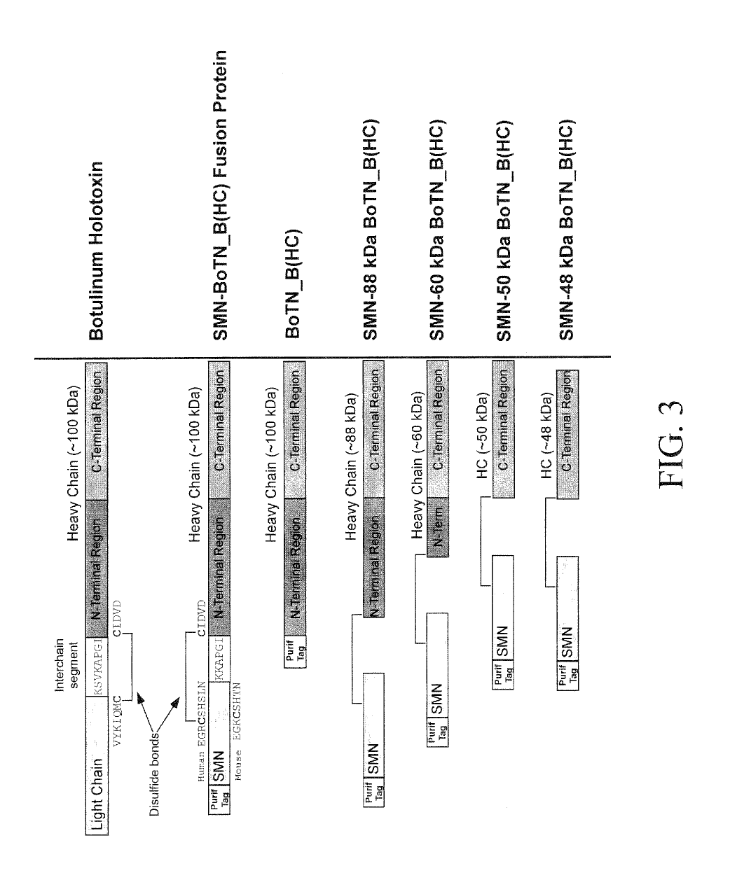

In one embodiment, the BoTN portion of a fusion protein of the invention is the BoTN heavy chain, or a fragment or variant thereof capable of providing for receptor-mediated endocytosis in a cell. A BoTN heavy chain portion of a fusion protein of the invention can be of any serotype, including A, B, C, D, E, F, or G. In one embodiment, the BoTN heavy chain portion is serotype A or B. In a specific embodiment, the BoTN heavy chain protein comprises the amino acid sequence shown in SEQ ID NO:3 or SEQ ID NO:4, or a fragment or variant thereof capable of providing for receptor-mediated endocytosis. In one embodiment, the BoTN portion can be the approximately 88 kDa, 60 kDa, 50 kDa, or 48 kDa fragment of BoTN heavy chain as shown in FIG. 3.

In another embodiment, the non-toxic BoTN portion comprises a modified and/or hybrid polypeptide that comprises amino acid sequences or polypeptides from non-BoTN (i.e., non-clostridial) proteins or polypeptides, and optionally BoTN polypeptides. Modified and/or hybrid polypeptide can provide one or more of (i) lacking the neurotoxin activities of botulinum and tetanus toxins, (ii) displaying high affinity to neuronal cells corresponding to the neuronal binding of tetanus neurotoxin, (iii) containing a domain which can effect translocation across cell membranes and (iv) having low affinity to neutralizing antibodies to tetanus toxin which are present as result of anti-tetanus inoculation. For example, in one embodiment, a non-toxic BoTN portion of the invention comprises a non-toxic portion of a diphtheria toxin and/or tetanus toxin. In one embodiment, a modified or hybrid polypeptide provides a cell binding and/or membrane translocation domain. In one embodiment, a non-toxic tetanus toxin comprises the tetanus toxin heavy chain (SEQ ID NO:7), or a fragment or variant thereof capable of providing for cell binding (e.g., neuron) and/or membrane translocation. In a further embodiment, a tetanus toxin portion is modified so as to reduce or eliminate immunogenic epitopes associated with tetanus toxin. In one embodiment, a non-toxic BoTN portion of a fusion protein of the invention comprises a translocation domain of diphtheria toxin (for example, amino acids 194-386) and the carboxy-terminal half of BoTN heavy chain into which domains of tetanus toxin having binding activity for cells have been inserted. In another embodiment, the non-toxic BoTN portion comprising a modified and/or hybrid polypeptide is modified so as to provide for reduced antibody response, reduced aggregation and/or increased solubility of the BoTN heavy chains in aqueous solution. Examples of non-toxic BoTN portions comprising modified and/or hybrid polypeptides contemplated within the scope of and that can be utilized in a fusion protein of the invention include, but are not limited to, those described in U.S. Pat. No. 7,368,532.

In one embodiment, the SMN polypeptide portion and the BoTN heavy chain portion of the fusion protein are connected via a disulfide bond. In another embodiment, the SMN polypeptide portion and the BoTN heavy chain portion are connected via a chemical moiety linking group. In another embodiment, the SMN polypeptide portion and the BoTN heavy chain portion are connected via an interchain amino acid segment or linker. In one embodiment, the interchain amino acid segment or linker comprises a protease cleavage site. In a still further embodiment, the SMN polypeptide portion and the BoTN heavy chain portion are connected via an interchain amino acid segment or linker and a disulfide bond between cysteine amino acids in the SMN and BoTN portions. In yet a further embodiment, the SMN and BoTN portions are directly connected wherein a terminal amino acid of the SMN portion is covalently bonded to a terminal amino acid of the BoTN portion. In a specific embodiment, the interchain amino acid segment or linker has the amino acid sequence KSVKAPGI (SEQ ID NO:11). In another specific embodiment, the interchain amino acid segment or linker has the amino acid sequence KKAPGI (SEQ ID NO:12). These interchain amino acid segments can be cleaved by trypsin. Other interchain amino acid segments and linkers are known in the art and include, but are not limited to, CGLVPAGSGP (SEQ ID NO:16) and CGLVPAGSGPSAGSSAC (SEQ ID NO:17). These interchain amino acid segments can be cleaved by thrombin protease.

In one embodiment, a fusion protein of the invention can be prepared wherein the SMN portion is incorporated within a liposome and the non-toxic BoTN portion is embedded in the liposome phospholipid bilayer or is outside of the liposome but tethered to the liposome and/or to the SMN protein via a chemical linker or moiety, wherein a cell binding portion or domain of the BoTN remains on the exterior of the lipid bilayer. In one embodiment, the chemical linker is an amino acid sequence that contains a hydrophobic sequence that can pass through or that is soluble in the lipid bilayer. In one embodiment, a non-toxic BoTN polypeptide comprises a transmembrane amino acid sequence and optionally a membrane anchor sequence. Examples of transmembrane and membrane anchor sequences are known in the art. In another embodiment, the SMN protein portion is encapsulated within the liposome but is not covalently attached to the non-toxic BoTN portion, which is tethered to the liposome via a chemical linker or moiety or is embedded within the bilayer wherein a cell binding portion or domain of the BoTN remains on the exterior of the lipid bilayer. In one embodiment, the non-toxic BoTN portion is tethered to the liposome via an alkyl group covalently bonded to the BoTN polypeptide. U.S. Pat. No. 6,159,931 describes linkage of a protein molecule to a long chain alkyl group via an amine linkage. The alkyl group can associate with the lipid bilayer of the liposome thereby tethering the protein molecule to the liposome. Methods for preparing liposomes and encapsulating proteins within the liposome are well known in the art.

The subject invention also concerns methods for treating, inhibiting the progression of, and/or preventing a disorder associated with and/or characterized by neuronal degeneration, such as SMA or TIPN, in a person or animal. In one embodiment, the neuronal degeneration of the disorder is characterized as motor axonal degeneration and/or sensory axonal degeneration. The neuronal degeneration in the person or animal can also be accompanied by impaired SMN axonal transport in neurons. In one embodiment, a therapeutically effective amount of a fusion protein or composition of the invention is administered to a person or animal in need of treatment. Administration can be continuous or at distinct intervals, as can be determined by a person of ordinary skill in the art. In one embodiment, the fusion protein is administered orally in liquid or solid form (e.g., as a tablet or capsule). Optionally, the fusion protein or composition can be administered in conjunction with HA and/or NTNH proteins, and/or with components of the SMN complex, and/or with proteins and compounds that promote SMN protein stability as described in Burnett et al. (2009). In one embodiment, a fusion protein of the invention can be administered in conjunction with glycosides.

For purposes of treating, inhibiting the progression of, and/or preventing TIPN, a fusion protein or composition of the invention may be administered prior to, during the course of, or after the person or animal has received treatment with a taxane compound, such as paclitaxel and/or docetaxel. Thus, the person or animal may already be suffering from TIPN, or in other cases, the person or animal may not yet have developed symptoms associated with TIPN. In one embodiment, a fusion protein or composition of the invention may be administered to the person or animal in conjunction with or at the same time as a taxane compound. Methods of the invention also contemplate that a fusion protein or composition of the invention can be administered in conjunction with other known drugs or treatments for TIPN or SMA.

For purposes of treating, inhibiting the progression of, and/or preventing SMA, a fusion protein or composition of the invention may be administered to a person or animal who already has SMA, or that is at risk of developing SMA. In one embodiment, the method also comprises genetic screening of the person or animal to determine their genetic status with regard to smn genes.

The subject invention also concerns methods for delivering or transporting a survival motor neuron protein to an axon terminal of a neuron. In one embodiment, a neuron is contacted with a fusion protein or composition of the invention. In one embodiment, the neuron is a mammalian neuron. In a specific embodiment, the neuron is a human neuron. In one embodiment, a fusion protein of the invention can be administered in conjunction with glycosides.

Compositions of the invention include a fusion protein of the invention. While a fusion protein of the invention can be administered as an isolated protein, these fusion proteins can also be administered as part of a pharmaceutical composition. In one embodiment, a composition of the invention comprises one or more fusion proteins in association with at least one pharmaceutically acceptable carrier and/or diluent. The pharmaceutical composition can be adapted for various routes of administration, such as oral, enteral, parenteral, intravenous, intramuscular, and so forth. Optionally, a composition of the invention can comprise a fusion protein of the invention along with HA and/or NTNH proteins of Clostridium, and/or with components of the SMN complex, and/or with proteins and compounds that promote SMN protein stability as described in Burnett et al. (2009).

The fusion proteins of the invention can be formulated according to known methods for preparing pharmaceutically useful compositions. Formulations are described in a number of sources which are well known and readily available to those skilled in the art. For example, Remington's Pharmaceutical Science (Martin 1995) describes formulations which can be used in connection with the subject invention. Formulations suitable for administration include, for example, aqueous sterile solutions, which may contain antioxidants, buffers, bacteriostats, and solutes; and aqueous and nonaqueous sterile suspensions which may include suspending agents and thickening agents. The formulations may be presented in unit-dose or multi-dose containers, for example sealed ampoules and vials, and may be stored in a freeze dried (lyophilized) condition requiring only the condition of the sterile liquid carrier, for example, water, prior to use. Extemporaneous injection solutions and suspensions may be prepared from sterile powder, granules, tablets, etc. It should be understood that in addition to the ingredients particularly mentioned above, the compositions of the subject invention can include other agents conventional in the art having regard to the type of formulation in question.

The fusion proteins of the present invention include all hydrates and salts that can be prepared by those of skill in the art. Under conditions where the fusion proteins of the present invention are sufficiently basic or acidic to form stable nontoxic acid or base salts, administration of the fusion protein salts may be appropriate. Examples of pharmaceutically acceptable salts are organic acid addition salts formed with acids that form a physiological acceptable anion, for example, tosylate, methanesulfonate, acetate, citrate, malonate, tartarate, succinate, benzoate, ascorbate, alpha-ketoglutarate, and alpha-glycerophosphate. Suitable inorganic salts may also be formed, including hydrochloride, sulfate, nitrate, bicarbonate, and carbonate salts.

Pharmaceutically acceptable salts of a fusion protein may be obtained using standard procedures well known in the art, for example, by reacting a sufficiently basic compound such as an amine with a suitable acid affording a physiologically acceptable anion. Alkali metal (i.e., sodium, potassium or lithium) or alkaline earth metal (i.e., calcium) salts of carboxylic acids can also be made.

Fusion proteins of the invention, and compositions thereof, may be systemically administered, such as orally or intravenously (optionally in combination with a pharmaceutically acceptable carrier such as an inert diluent), or an assimilable edible carrier for oral delivery. They may be enclosed in hard or soft shell gelatin capsules, compressed into tablets, or incorporated directly with the food of the patient's diet. For oral therapeutic administration, the active compound may be combined with one or more excipients and used in the form of ingestible tablets, buccal tablets, troches, capsules, elixirs, suspensions, syrups, wafers, aerosol sprays, and the like.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac, or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

Useful dosages of the fusion proteins and pharmaceutical compositions of the present invention can be determined by comparing their in vitro activity, and in vivo activity in animal models. Methods for the extrapolation of effective dosages in mice, and other animals, to humans are known to the art; for example, see U.S. Pat. No. 4,938,949.

The dose administered to a patient, particularly a human, in the context of the present invention should be sufficient to achieve a therapeutic response in the patient over a reasonable time frame, without lethal toxicity, and preferably causing no more than an acceptable level of side effects or morbidity. One skilled in the art will recognize that dosage will depend upon a variety of factors including the condition (health) of the subject, the body weight of the subject, the kind of concurrent treatment (if any), frequency of treatment, therapeutic ratio, as well as the severity and stage of the pathological condition.

To provide for the administration of such dosages for the desired therapeutic treatment, in some embodiments, pharmaceutical compositions of the invention can comprise between about 0.1% and 45%, and especially, 1 and 15%, by weight of the total of one or more of the compounds based on the weight of the total composition including carrier or diluents. Illustratively, dosage levels of the administered active ingredients can be: orally 0.01 to about 200 mg/kg, and preferably about 1 to 100 mg/kg; intravenous, 0.01 to about 20 mg/kg; intraperitoneal, 0.01 to about 100 mg/kg; subcutaneous, 0.01 to about 100 mg/kg; intramuscular, 0.01 to about 100 mg/kg; intranasal instillation, 0.01 to about 20 mg/kg; and aerosol, 0.01 to about 20 mg/kg of animal (body) weight.

The subject invention also concerns kits comprising one or more fusion proteins and/or compositions of the invention in one or more containers. In one embodiment, a kit of the invention comprises a fusion protein comprising human SMN1 protein (SEQ ID NO:1) or a fragment or variant thereof having SMN biological activity, and/or a BoTN heavy chain portion comprising the amino acid sequence of SEQ ID NO:3 or SEQ ID NO:4, or a fragment or variant thereof capable of providing for receptor-mediated endocytosis in a cell. In a more specific embodiment, the fusion protein comprises the amino acid sequence shown in any of SEQ ID NO:13, SEQ ID NO:14, or SEQ ID NO:15. Kits of the invention can optionally include pharmaceutically acceptable carriers and/or diluents. In one embodiment, a kit of the invention includes one or more other components, adjuncts, or adjuvants as described herein. In one embodiment, a kit of the invention includes instructions or packaging materials that describe how to administer a compound or composition of the kit. Containers of the kit can be of any suitable material, e.g., glass, plastic, metal, etc., and of any suitable size, shape, or configuration. In one embodiment, a compound and/or composition of the invention is provided in the kit as a solid, such as a tablet, pill, or powder form. In another embodiment, a compound and/or composition of the invention is provided in the kit as a liquid or solution. In one embodiment, the kit comprises an ampoule or syringe containing a compound and/or composition of the invention in liquid or solution form.

Mammalian species which benefit from the disclosed methods include, but are not limited to, primates, such as apes, chimpanzees, orangutans, humans, monkeys; domesticated animals (e.g., pets) such as dogs, cats, guinea pigs, hamsters, Vietnamese pot-bellied pigs, rabbits, and ferrets; domesticated farm animals such as cows, buffalo, bison, horses, donkey, swine, sheep, and goats; exotic animals typically found in zoos, such as bear, lions, tigers, panthers, elephants, hippopotamus, rhinoceros, giraffes, antelopes, sloth, gazelles, zebras, wildebeests, prairie dogs, koala bears, kangaroo, opossums, raccoons, pandas, hyena, seals, sea lions, elephant seals, otters, porpoises, dolphins, and whales. As used herein, the terms “patient” and “subject” are used interchangeably and are intended to include such human and non-human species.

Polypeptide variants having substitution of amino acids other than those specifically exemplified in the subject polypeptides are also contemplated within the scope of the present invention. For example, non-natural amino acids can be substituted for the amino acids of a polypeptide of the invention, so long as the polypeptide having substituted amino acids retains substantially the same activity as the polypeptide in which amino acids have not been substituted. Examples of non-natural amino acids include, but are not limited to, ornithine, citrulline, hydroxyproline, homoserine, phenylglycine, taurine, iodotyrosine, 2,4-diaminobutyric acid, a-amino isobutyric acid, 4-aminobutyric acid, 2-amino butyric acid, γ-amino butyric acid, e-amino hexanoic acid, 6-amino hexanoic acid, 2-amino isobutyric acid, 3-amino propionic acid, norleucine, norvaline, sarcosine, homocitrulline, cysteic acid, t-butylglycine, t-butylalanine, phenylglycine, cyclohexylalanine, β-alanine, fluoro-amino acids, designer amino acids such as β-methyl amino acids, C-methyl amino acids, N-methyl amino acids, and amino acid analogues in general. Non-natural amino acids also include amino acids having derivatized side groups. Furthermore, any of the amino acids in the protein can be of the D (dextrorotary) form or L (levorotary) form.

Amino acids can be generally categorized in the following classes: non-polar, uncharged polar, basic, and acidic. Conservative substitutions whereby a polypeptide having an amino acid of one class is replaced with another amino acid of the same class fall within the scope of the subject invention so long as the polypeptide having the substitution still retains substantially the same biological activity as a polypeptide that does not have the substitution. Table 1 below provides a listing of examples of amino acids belonging to each class.

TABLE 1

| Class of Amino Acid | Examples of Amino Acids |
|---|---|
| Nonpolar | Ala, Val, Leu, Ile, Pro, Met, Phe, Trp |
| Uncharged Polar | Gly, Ser, Thr, Cys, Tyr, Asn, Gln |
| Acidic | Asp, Glu |
| Basic | Lys, Arg, His |

The polypeptides of the present invention can be formulated into pharmaceutically-acceptable salt forms. Pharmaceutically-acceptable salt forms include the acid addition salts and include hydrochloric, hydrobromic, nitric, phosphoric, carbonic, sulphuric, and organic acids like acetic, propionic, benzoic, succinic, fumaric, mandelic, oxalic, citric, tartaric, maleic, and the like. Pharmaceutically-acceptable base addition salts include sodium, potassium, calcium, ammonium, and magnesium salts. Pharmaceutically-acceptable salts of the polypeptides of the invention can be prepared using conventional techniques.

The subject invention also concerns polynucleotides that encode the polypeptides of the invention and their use in the methods of the present invention. Methods and materials for synthesizing and preparing a polynucleotide encoding a polypeptide of the invention are well known in the art. Because of the degeneracy of the genetic code, a variety of different polynucleotide sequences can encode a peptide of the present invention. In addition, it is well within the skill of a person trained in the art to create alternative polynucleotide sequences encoding the same, or essentially the same, polypeptides of the subject invention. These variant or alternative polynucleotide sequences, and the polypeptides encoded thereby, are within the scope of the subject invention. As used herein, references to “essentially the same” sequence refers to sequences which encode amino acid substitutions, deletions, additions, and/or insertions which do not materially alter the functional activity of the polypeptide encoded by the polynucleotides of the present invention. Variant polypeptides having amino acid substitutions, deletions, additions, and/or insertions which do not materially alter the functional activity of the polypeptide can also be prepared using standard techniques known in the art, and such variant polypeptides are encompassed within the scope of the present invention.

The subject invention also concerns polynucleotide expression constructs that comprise a polynucleotide of the present invention comprising a nucleotide sequence encoding a polypeptide of the present invention. In one embodiment, the polynucleotide encodes a polypeptide comprising the amino acid sequence shown in SEQ ID NO:1 and/or SEQ ID NO:4, or a fragment or variant thereof. In a specific embodiment, the polynucleotide encodes a polypeptide comprising the amino acid sequence shown in any of SEQ ID NO:13, SEQ ID NO:14, or SEQ ID NO:15. A polynucleotide of the invention can optionally comprise a nucleotide sequence that encodes a peptide tag that can be used in purifying the protein produced when the polynucleotide is expressed. Examples of peptide tags include, but are not limited to, His6, S peptide, T7 peptide, calmodulin binding peptide, and maltose binding peptide. In one embodiment, the peptide tag is a FLAG-tag or FLAG octapeptide. In a specific embodiment, the peptide tag has the sequence DYKDDDDK (SEQ ID NO:10).

As used herein, the term “expression construct” refers to a combination of nucleic acid sequences that provides for transcription of an operably linked nucleic acid sequence. As used herein, the term “operably linked” refers to a juxtaposition of the components described wherein the components are in a relationship that permits them to function in their intended manner. In general, operably linked components are in contiguous relation.

Expression constructs of the invention will also generally include regulatory elements that are functional in the intended host cell in which the expression construct is to be expressed. Thus, a person of ordinary skill in the art can select regulatory elements for use in, for example, bacterial host cells, yeast host cells, plant host cells, insect host cells, mammalian host cells, and human host cells. Regulatory elements include promoters, transcription termination sequences, translation termination sequences, enhancers, and polyadenylation elements.

An expression construct of the invention can comprise a promoter sequence operably linked to a polynucleotide sequence encoding a polypeptide of the invention. Promoters can be incorporated into a polynucleotide using standard techniques known in the art. Multiple copies of promoters or multiple promoters can be used in an expression construct of the invention. In a preferred embodiment, a promoter can be positioned about the same distance from the transcription start site as it is from the transcription start site in its natural genetic environment. Some variation in this distance is permitted without substantial decrease in promoter activity. A transcription start site is typically included in the expression construct.

For expression in animal cells, an expression construct of the invention can comprise suitable promoters that can drive transcription of the polynucleotide sequence. If the cells are mammalian cells, then promoters such as, for example, actin promoter, metallothionein promoter, NF-kappaB promoter, EGR promoter, SRE promoter, IL-2 promoter, NFAT promoter, osteocalcin promoter, SV40 early promoter and SV40 late promoter, Lck promoter, BMP5 promoter, TRP-1 promoter, murine mammary tumor virus long terminal repeat promoter, STAT promoter, or an immunoglobulin promoter can be used in the expression construct. The baculovirus polyhedrin promoter can be used with an expression construct of the invention for expression in insect cells. Promoters suitable for use with an expression construct of the invention in yeast cells include, but are not limited to, 3-phosphoglycerate kinase promoter, glyceraldehyde-3-phosphate dehydrogenase promoter, metallothionein promoter, alcohol dehydrogenase-2 promoter, and hexokinase promoter.

For expression in prokaryotic systems, an expression construct of the invention can comprise promoters such as, for example, alkaline phosphatase promoter, tryptophan (trp) promoter, lambda P_{L} promoter, β-lactamase promoter, lactose promoter, phoA promoter, T3 promoter, T7 promoter, or tac promoter (de Boer et al., 1983).

If the expression construct is to be provided in a plant cell, plant viral promoters, such as, for example, the cauliflower mosaic virus (CaMV) 35S (including the enhanced CaMV 35S promoter (see, for example U.S. Pat. No. 5,106,739)) or 19S promoter can be used. Plant promoters such as prolifera promoter, Ap3 promoter, heat shock promoters, T-DNA 1′- or 2′-promoter of A. tumafaciens, polygalacturonase promoter, chalcone synthase A (CHS-A) promoter from petunia, tobacco PR-1a promoter, ubiquitin promoter, actin promoter, alcA gene promoter, pin2 promoter (Xu et al., 1993), maize WipI promoter, maize trpA gene promoter (U.S. Pat. No. 5,625,136), maize CDPK gene promoter, and RUBISCO SSU promoter (U.S. Pat. No. 5,034,322) can also be used. Seed-specific promoters such as the promoter from a β-phaseolin gene (of kidney bean) or a glycinin gene (of soybean), and others, can also be used. Constitutive promoters (such as the CaMV, ubiquitin, actin, or NOS promoter), tissue-specific promoters (such as the E8 promoter from tomato), developmentally-regulated promoters, and inducible promoters (such as those promoters than can be induced by heat, light, hormones, or chemicals) are contemplated for use with the polynucleotides of the invention.

Expression constructs of the invention may optionally contain a transcription termination sequence, a translation termination sequence, signal peptide sequence, and/or enhancer elements. Transcription termination regions can typically be obtained from the 3′ untranslated region of a eukaryotic or viral gene sequence. Transcription termination sequences can be positioned downstream of a coding sequence to provide for efficient termination. Signal peptides are a group of short amino terminal sequences that encode information responsible for the relocation of an operably linked peptide to a wide range of post-translational cellular destinations, ranging from a specific organelle compartment to sites of protein action and the extracellular environment. Targeting a peptide to an intended cellular and/or extracellular destination through the use of operably linked signal peptide sequence is contemplated for use with the fusion proteins of the invention. Chemical enhancers are cis-acting elements that increase gene transcription and can also be included in the expression construct. Chemical enhancer elements are known in the art, and include, but are not limited to, the CaMV 35S enhancer element, cytomegalovirus (CMV) early promoter enhancer element, and the SV40 enhancer element. DNA sequences which direct polyadenylation of the mRNA encoded by the structural gene can also be included in the expression construct.

Unique restriction enzyme sites can be included at the 5′ and 3′ ends of the expression construct to allow for insertion into a polynucleotide vector. As used herein, the term “vector” refers to any genetic element, including for example, plasmids, cosmids, chromosomes, phage, virus, and the like, which is capable of replication when associated with proper control elements and which can transfer polynucleotide sequences between cells. Vectors contain a nucleotide sequence that permits the vector to replicate in a selected host cell. A number of vectors are available for expression and/or cloning, and include, but are not limited to, pBR322, pUC series, M13 series, and pBLUESCRIPT vectors (Stratagene, La Jolla, Calif.).

Polynucleotides and polypeptides of the subject invention can also be defined in terms of more particular identity and/or similarity ranges with those exemplified herein. The sequence identity will typically be greater than 60%, preferably greater than 75%, more preferably greater than 80%, even more preferably greater than 90%, and can be greater than 95%. The identity and/or similarity of a sequence can be 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% as compared to a sequence exemplified herein. Unless otherwise specified, as used herein percent sequence identity and/or similarity of two sequences can be determined using the algorithm of Karlin and Altschul (1990), modified as in Karlin and Altschul (1993). Such an algorithm is incorporated into the NBLAST and XBLAST programs of Altschul et al. (1990). BLAST searches can be performed with the NBLAST program, score=100, wordlength=12, to obtain sequences with the desired percent sequence identity. To obtain gapped alignments for comparison purposes, Gapped BLAST can be used as described in Altschul et al. (1997). When utilizing BLAST and Gapped BLAST programs, the default parameters of the respective programs (NBLAST and XBLAST) can be used (See NCBI/NIH website).

The subject invention also contemplates those polynucleotide molecules (encoding polypeptides of the invention) having sequences which are sufficiently homologous with the polynucleotide sequences exemplified herein so as to permit hybridization with that sequence under standard stringent conditions and standard methods (Maniatis et al., 1982). As used herein, “stringent” conditions for hybridization refers to conditions wherein hybridization is typically carried out overnight at 20-25 C below the melting temperature (Tₘ) of the DNA hybrid in 6×SSPE, 5×Denhardt's solution, 0.1% SDS, 0.1 mg/ml denatured DNA. The melting temperature is described by the following formula (Beltz et al., 1983):

Tₘ=81.5° C.+16.6 Log [Na+]+0.41(% G+C)−0.61(% formamide)−600/length of duplex in base pairs.

Washes are typically carried out as follows:

(1) Twice at room temperature for 15 minutes in 1×SSPE, 0.1% SDS (low stringency wash).

(2) Once at Tₘ20° C. for 15 minutes in 0.2×SSPE, 0.1% SDS (moderate stringency wash).

As used herein, the terms “nucleic acid” and “polynucleotide sequence” refer to a deoxyribonucleotide or ribonucleotide polymer in either single- or double-stranded form, and unless otherwise limited, would encompass known analogs of natural nucleotides that can function in a similar manner as naturally-occurring nucleotides. The polynucleotide sequences include both the DNA strand sequence that is transcribed into RNA and the RNA sequence that is translated into protein. The polynucleotide sequences include both full-length sequences as well as shorter sequences derived from the full-length sequences. It is understood that a particular polynucleotide sequence includes the degenerate codons of the native sequence or sequences which may be introduced to provide codon preference in a specific host cell. The polynucleotide sequences falling within the scope of the subject invention further include sequences which specifically hybridize with the exemplified sequences. The polynucleotide includes both the sense and antisense strands as either individual strands or in the duplex.

The subject invention also concerns packaged dosage formulations comprising in one or more containers a fusion protein or composition of the subject invention formulated in a pharmaceutically acceptable dosage. The package can contain discrete quantities of the dosage formulation, such as tablet, capsules, lozenge, and powders. The quantity of fusion protein or composition in a dosage formulation and that can be administered to a patient can vary from about 1 mg to about 2000 mg, more typically about 1 mg to about 500 mg, or about 5 mg to about 250 mg, or about 10 mg to about 100 mg. In one embodiment, a packaged dosage formulation also comprises one or more taxane compounds, such as paclitaxel and/or docetaxel.

Compounds and compositions of the invention can be provided as an oral medication, e.g., either as a liquid or solid, e.g., a tablet. The solid form can optionally comprise an enteric coating that prevents it from dissolving until it reaches the small intestine. In one embodiment, the formulation in the medication contains SMN-BoTN_B(HC) fusion proteins, optionally with or without the auxiliary HA/NTNH proteins and optionally with or without other proteins in the SMN complex and/or proteins or compounds that otherwise stabilize SMN. The formulation can also optionally contain additional ingredients as described in U.S. Published Application No. 2004/0013687 and Burnett et al. (2009), and components of the SMN complex.

The invention applies to mouse and human systems. SMN is predicted to have post-translational modifications that cannot be provided by E. coli cells, so typically the protein is expressed in mammalian cells, e.g., mouse or human cells.

### Materials and Methods

It has been demonstrated in vitro in U.S. Published Application No. 2004/0013687 that native and truncation mutants of the BoTN heavy chain (serotypes A and B) are capable of transcytosing a variety of molecules across polarized human intestinal epithelial cells. Truncation mutants were designed to contain the minimal components required for binding and endocytosis and minimize interfering immune responses (see FIG. 15 of U.S. Published Application No. 2004/0013687). U.S. Published Application No. 2004/0013687 also demonstrates in vivo that orally administered native BoTN heavy chain A was absorbed by the intestinal epithelium and transcytosed to the blood stream which produced an immune response in mice (U.S. Published Application No. 2004/0013687). Pages 172-176 of U.S. Published Application No. 2004/0013687 list in detail possible formulations for oral and topical administration.

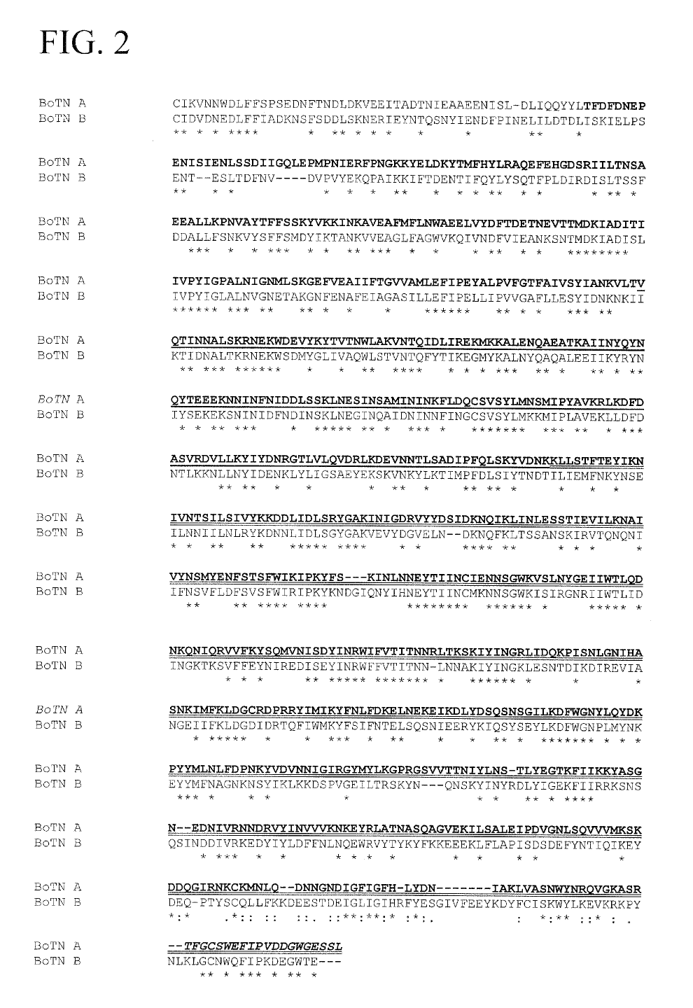

An assay to demonstrate neuronal endocytosis of tetanus toxin fragments linked to other molecules has previously been described (Francis et al., 2004). While a SOD1-TeTN fusion protein was successfully endocytosed, attempts with SMN failed (see FIGS. 4A and 4B of Francis et al., 2004). It was concluded that endocytosis of the SMN-tetanus toxin heavy chain fragment fusion protein they tested was due to some unidentified feature of the SMN moiety. Zhang et al. (2003) describe an experiment to demonstrate SMN axonal transport (see FIG. 3 of Zhang et al. (2003)). Peters et al. (2007) and Jimenez-Andrade (2006) describe experiments to determine the pathology of TIPN (see FIG. 3 of Peters et al. 2007). Pellizzoni et al. (2002) describe a recombinant plasmid containing the SMN gene fused to a purification tag for expression and purification of human SMN in human cell lines (see FIG. 2 of Pellizzoni et al. (2002)). Methods to detect the presence or absence of SMN protein in tissues and the pathology of SMA type II model mice have been described in Grondard et al. (2005) (see FIG. 3 of Grondard et al. (2005)).

### DNA Constructs

Recombinant botulinum neurotoxin B heavy chain (BoTN_B (HC)). With standard DNA isolation techniques and methods, minimal fragments of the BoTN_B(HC) optionally with modified or hybrid polypeptides required for binding and endocytosis containing an N-terminal purification tag are subcloned into an expression vector appropriate for use in bacterial expression systems. Examples of constructs are shown in FIG. 3.

Recombinant survival motor neuron-botulinum neurotoxin B heavy chain (SMN-BoTN_B(HC)) fusion protein. Any post translational modifications for SMN are available in mammalian cells and the BoTN_B(HC) portion is correctly expressed since codon bias does not present a problem. A fusion protein similar to the WT holotoxin promotes specific formation of the critical disulfide bond.

With mouse cDNA, PCR, site-directed mutagenesis and other standard recombinant DNA techniques, along with methods of BoTN gene isolation, the gene sequence of an N-terminal purification peptide tag followed by the mouse smn gene fused to the BoTN_B(HC) (and its truncation mutants) are together subcloned into an expression vector appropriate for use in cell lines for large scale expression as deemed appropriate. Examples of constructs are shown in FIG. 3.

All constructs are verified by DNA sequencing.

### Cell Culture and Protein Expression

Constructs containing the SMN polypeptide portion—Protein expression is optimized for large scale expression of soluble tagged SMN protein. Employed expression systems include bacterial cells, insect cells and mammalian cells.

Constructs containing only the BoTN heavt chain—Expression is performed in E. coli strain BL21 codon plus (DE3)-RIL (Stratagene). Cultures are grown in Lennox broth at 37° C., with shaking, to an O.D. at 600 nanometers of 0.6 to 0.8. Isopropyl-beta-D-thiogalactopyranoside (IPTG) is added to 1.0 mM (final concentration), and incubation is continued for an additional 6+ hours.

### Purification

Constructs containing SMN—Total cell extracts are prepared by resuspending cell pellets in optimized solution near physiological pH containing a buffering agent, NaCl, MgCl₂, 0.1% non-ionic detergent, and protease inhibitors. Following centrifugation at 10,000 rpm for 15 min, supernatants are passed through a 0.2-μm filter and added to affinity purification beads pre-washed with the same buffer. Extracts are incubated with these beads for 2 h at 4° C. Supernatants are discarded, and beads are extensively washed with the resuspension buffer containing 0.02% non-ionic detergent. Three high salt washes are performed with ten bed volumes of resuspension buffer containing at least 500 mM NaCl and 0.02% non-ionic detergent for 15 min at 4° C. After the next three low salt washes with resuspension buffer containing 0.02% nonionic detergent, bound complexes are separated from the beads with 10 bed volumes of the same buffer containing high concentrations of eluent peptides or compounds for 1 h at 4° C. (Pellizzoni et al., 2002).

Because this method purifies the native SMN complex (Pellizzoni et al., 2002), an alternative strategy can be used to obtain higher levels of homogenous fusion protein if desired: Express the fusion protein in bacteria as described below, with adjustments to buffers as needed. Prepare mammalian cell extracts by incubating them with antibody affinity beads that pull down the other proteins in the SMN complex. The remaining components of the cell extract are sufficient to provide the putative post-translational modifications. This extract containing the bacterially expressed and modified fusion protein can then be further purified as described above.

Constructs containing only the BoTN heavy chain—Bacteria from 1 liter of induced culture are harvested by centrifugation at 4° C. and re-suspended in 20 mL of 50 mM sodium phosphate buffer (pH. 7.4) with 300 mM NaCl. The cell suspension is lysed on ice by sonication, with two pulses of 1 minute each at 75% power, with a model 60 sonic dismembrator (Thermo-Fisher). Lysates are centrifuged at 20000×g for 30 minutes at 4° C. The clarified supernatants are mixed with 2 mL of packed nitriletriacetic resin, incubated for one hour at 4° C. on a rotator, and finally poured into a 25-mL column. The column is washed with 30 volumes of washing buffer (50 mM sodium phosphate (pH 6.0), 300 mM NaCl, 25 mM imidazole). Bound proteins are eluted with elution buffer (50 mM sodium phosphate (pH 4.5), 300 mM NaCl, 350 mM imidazole). Purified proteins are analyzed by sodium dodecyl sulfate-polyacrylamide gel electrophoresis (SDS-PAGE) and Western blotting (as described in U.S. Published Application No. 2004/0013687).

### Functional Assays to Confirm Protein Activity

Nicking and Reducing the SMN-BoTN_B(HC) fusion protein (see, for example, U.S. Published Application No. 2004/0013687)—This enables the separation of the two protein moieties for individual testing. Botulinum toxin is expressed as a relatively inactive single chain molecule. To become fully active, the toxin must undergo proteolytic processing (“nicking”) to yield its dichain form. In the laboratory, this is typically accomplished with trypsin and the same procedure is used to separate SMN from the BoTN heavy chain.

In order to facilitate the subsequent separation of proteins from the nicking enzyme, TPCK (L-1-tosylamido-2-phenylethyl chloromethyl ketone) treated trypsin cross-linked to 4% beaded agarose is used (Immobilized Trypsin; PIERCE). The trypsin slurry is washed 3 times with reaction buffer (10 mM Sodium Phosphate Buffer, pH 7.5). Protein is added and incubated with enzyme at room temperature (23° C.) for one hour at a 1:10 ratio of trypsin to protein. After incubation, the reaction mixture is centrifuged at 10,000 rotations per minute in an Eppendorf tabletop centrifuge for 5 minutes. The supernatant containing “nicked” protein is collected and stored at −20° C. Alternatively, “nicked” protein can be separated from the beaded trypsin by filtration through a 0.2 micron centrifugal filter (Schleicher & Schuell Centrex Microfilter Unit) into a clean, sterile tube. A sample of the material is examined by electrophoresis to verify nicking.

The dichain fusion protein consists of SMN and the BoTN heavy chain linked by a disulfide bond. This bond must be reduced (broken) for the proteins to demonstrate their relative activities.

The fusion protein is reduced by incubating it with dithiothreitol (DTT; Cleland's Reagent) in phosphate buffer at physiological pH (pH 7.2-7.4) or in phosphate buffered saline (PBS). The concentration of DTT typically used is 5 mM to 20 mM, depending on the experiment. The DTT and protein reaction mixture is incubated at room temperature (23° C.) for one hour. Disulfide bond reduction is verified by electrophoresis on non-reducing gels.

SMN binding—A biotinylated synthetic peptide encoding amino acids 300-324 (RGRGRGGFDRGGMSRG-GRGGGRGGM) (SEQ ID NO:9) of Ewings sarcoma protein (Sigma) is used to assess whether separated SMN and the SMN moiety of the fusion protein possesses RG/RGG domain binding activity in vitro. This RG peptide has previously been shown to interact directly with recombinant full-length SMN. One ng of the RG peptide is immobilized on a streptavidin BIAcore chip corresponding to a baseline increase of 1000 RU (1 RU represents 1 pg of bound sample). The immobilized peptide is then pulsed with 10 μg of either SMN, BoTN_B (HC), or the fusion protein. In control experiments, recombinant peptides encoded by SMN exons 1 and 4 are used to determine nonspecific background binding to the synthetic RG peptide. A control reference chip lacking the RG peptide is also used to determine nonspecific binding to the streptavidin chip itself. All experiments are repeated in triplicate (Francis et al., 2004).

BoTN_B(HC) Transcytosis (see, for example, U.S. Published Application No. 2004/0013687)—Monolayers of polarized epithelial cells are grown on polycarbonate membranes with a 0.4 micrometer pore size in TRANSWELL (Corning-Costar) porous bottom inserts. The TRANSWELL apparatus permits containment of a product on either the apical or basolateral face of an epithelial cell culture. In the absence of transcytosis of the product across the epithelial cell layer, substantially all of the product is retained on one side of the epithelium by the apparatus. The TRANSWELL apparatus is therefore useful for assessing transepithelial transcytosis of products.

The cell growth area within each TRANS WELL insert is equivalent to one square centimeter. Prior to seeding cells, insert membranes are coated with 10 μg per square centimeter rat tail type I collagen. Collagen stock solution (6.7 mg per mL) is prepared in sterile 1% (v/v) acetic acid and stored at 3° C. This collagen stock solution is diluted, as needed, in ice cold 60% (v/v) ethanol, and 150 μL of the resulting solution containing 10 micrograms of diluted collagen is added to each well.

The collagen solution is allowed to dry at room temperature overnight (about eighteen hours). After drying, the wells are sterilized under UV light for one hour, followed by a pre-incubation with cell culture medium (thirty minute incubation). The pre-incubation medium is removed immediately prior to addition of cells and fresh medium. Cells are plated in the TRANSWELL apparatus at confluent density. The volumes of medium added are 0.5 mL to the upper chamber and 1.0 mL to the bottom chamber. Culture medium is changed every two days. The cultures maintained in twelve-well plates are allowed to differentiate a minimum of ten days before use. The integrity of cell monolayers and formation of tight junctions are visualized by monitoring the maintenance of a slightly higher medium meniscus in the inserts as compared to the bottom wells. Formation of tight junctions are confirmed experimentally by assaying the rate of (³H)-inulin diffusion from the top well into the bottom chamber or by measurement of transepithelial resistance across the monolayer.

Transcytosis is assayed by replacement of medium, usually in the top well, with an appropriate volume of medium containing various concentrations of (¹²⁵I-labeled proteins of interest, separated BoTN_B(HC) and the fusion protein. Transport of radiolabeled protein is monitored by sampling the entire content of opposite wells, which is usually the bottom wells. Aliquots (0.5 μL) of the sampled medium are filtered through a SEPHADEX™ G-25 column (GE Healthcare), and 0.5 mL fractions are collected. The amount of radioactivity in the fractions is determined using a gamma counter. The amount of transcytosed protein is normalized and expressed as femtomoles per hour per square centimeter of cultured cell surface. A minimum of two replicates per condition are included in each experiment, and experiments are typically reproduced at least three times.

### Basic Endocytosis Assay

Cell Culture—NSC 19 cells or other neuronal cells as appropriate are grown in Dulbecco's modified Eagle's medium (DMEM) supplemented with 10% fetal calf serum, and transferred to DMEM supplemented with 0.5 g/L insulin, 0.5 g/L transferrin, 0.5 mg/L sodium selenite, 1.6 g/L putrescine, and 0.73 mg/L progesterone (N1 medium for neural cells; Sigma) as required. Cells are maintained in a humidified incubator with 5% CO₂.

Immunofluorescence and Confocal Imaging—NSC19 cells or other neuronal cells as appropriate are seeded onto 0.1 mg/ml poly-L-lysine-coated 13-mm coverslips, using normal growth media. Direct immunofluorescence is used to study the cellular localization of the fusion protein following its incubation with cultured cells. Neurons are incubated with either medium alone, fusion protein, reduced peptide-tagged SMN or His-tagged BoTN at optimized concentrations in serum-free media for optimized time frames. The medium is removed and cells are incubated with mouse monoclonal antibody specific for the peptide tag in the SMN construct and conjugated to a fluorophore that emits in the red region of the visible spectrum, or mouse monoclonal anti-BoTN-FITC (Genovac) (emits in the green region of the visible spectrum) diluted as needed in 1% (w/v) bovine serum albumin/DMEM for required times at 4° C. Labeled anti-bodies can also be applied simultaneously. The cells are washed twice in PBS and fixed using 4% (w/v) paraformaldehyde in PBS for 30 min at room temperature. The cells are again washed twice with PBS and the coverslips are mounted in Vectashield mounting media (Vector Labs) and evaluated for signs of fusion protein staining and toxicity by confocal microscopy using a Zeiss LSM410 confocal laser-scanning microscope with a krypton-argon laser and a plan-apochromat 63× objective lens with a 1.4 numeric aperature. Digitized images were then processed using Adobe Photoshop 5.0 software.

### Axonal Transport Assay

Enhanced green fluorescent protein reporter constructs and neuron transfection. Full-length cDNA of the human SMN1 is subcloned into an enhanced green fluorescent protein (EGFP)-C1 vector (BD Biosciences Clontech) and is designated EGFP-SMN (Zhang et al., 2003). The construct is sequenced to ensure that no frame shift occurred. This provides a way to observe an endogenously expressed form of SMN protein.

Cultured neurons are transfected with EGFP-SMN DNA using DOTAP liposomal reagent (Roche) and cultured for 4 d, as described in Zhang et al., 2003. The cells are fixed in 4% paraformaldehyde for 20 min at room temperature. Images are captured using a cooled CCD camera with a fluorescence microscope. For live cell imaging, transfected neurons were grown on Bioptechs coverslips (40 mm) for 4 days after transfection (described below).

Cell culture (Zhang et al., 2003)—Rat spinal cords (E15) are dissected, and ventral regions are cut into small pieces and trypsinized (0.1% in HBSS) at 37° C. for 10 min. The tissues are gently dissociated by triturating in minimal essential medium (MEM) with 10% FBS (Sigma). Large motor neurons are harvested by density gradient centrifugation through 6.8% metrizamide cushion in Leibovitz's L-15 medium (Invitrogen) at 500×g for 10 min. After washing twice in MEM, the cells are plated at low density (5000 cells/cm²) on poly-D-lysine (25 g/ml, 16 hr) and laminin A (0.02 mg/ml, 12 min) coated coverslips in MEM with 10% FBS for 2 hr. Cells are inverted onto a monolayer of rat astrocytes in N₃-conditioned medium with 0.5% FBS, 10 ng/ml NGF, 25 ng/ml NT-3, and 25 ng/ml BDNF, and cultured for 3 d at 37° C. in 5% CO₂. N₃-conditioned medium contained MEM supplemented with transferrin (0.2%), ovalbumin (0.1%), insulin (10 g/ml), putrescine (32 g/ml), sodium selenite (26 ng/ml), progesterone (12.5 ng/ml), hydrocortisone (9.1 ng/ml), T3 (3,3′,5′-tri-iodo-Lthyronine, sodium salt, 20 ng/ml), and BSA (10 g/ml). For immunofluorescence analysis as described in the previous section, cells are fixed in paraformaldehyde (4% in 1×PBS) for 20 min at room temperature and washed in 1×PBS with 5 mM MgCl₂ three times.

In initial controls, neurons are treated with the exogenous tagged SMN protein construct alone to determine its therapeutic effectiveness. Methods of delivery include but are not limited to microinjection, adenoviral vectors and lentiviral vectors. As needed, cell cultures will be probed with antibodies to reveal the localization of the exogenous SMN construct. This experiment determines that the SMN construct is localized to axons, which is an indicator of proper function. The experiment is repeated in neurons cultured from SMA model mice. Rescue of axon terminal defects in these SMA neurons demonstrate the therapeutic effectiveness of the SMN construct.

Control experiments are followed by neuronal cultures treated separately with fusion protein or paclitaxel at optimized concentrations in medium for required time frames and the effects on EGFP-SMN transport and axonal defects are analyzed. For paclitaxel treatments, the concentrations needed for four levels of transport inhibition, ranging from low to high, are determined. For fusion protein treatment, the maximum concentration having no effect on EGFP-SMN transport is established as optimal. In test sets, initially healthy neurons are treated first with paclitaxel at four concentrations and the results on transport are noted. Second, fusion protein is applied to the same cells at the optimal concentration for the required times and the results are analyzed. Controls using delivery medium only are done in parallel. Live cell imaging is used in these experiments. Immunofluorescence techniques are used for evaluating SMA mouse neurons, in which preliminary axonal defects are measured and documented, fusion protein is applied at various concentrations and its affects on axonal repair are observed over time.

Fluorescence microscopy and digital imaging—Neurons are visualized using a Nikon Eclipse inverted microscope equipped with a 60× Plan-Neofluar objective, phase optics, 100 W mercury arc lamp, and HiQ bandpass filters (Chroma Technology, Brattleboro, Vt.). Images are captured with a cooled CCD camera (Quantix; Photometrics) using a 35 mm shutter and processed using IP Lab Spectrum (Scanalytics). Fluorescence images of proteins (immunofluorescence) are then acquired with specific filters, including Cy2, Cy3, or Cy5. Exposure time is kept constant and below gray scale saturation. Quantitative analysis of neurite length in each transfection is completed on phase optics from duplicated coverslips. The longest neurites from more than 60 transfected neurons are measured using computer IP Lab software.

For live cell imaging as described in Zhang et al., 2003, neurons are transfected with an EGFP-SMN construct and cultured on Bioptechs coverslips (40 mm) for 4 d in N₃-conditioned medium. Coverslips are transferred to a sealed environmental chamber (Bioptechs Focht Chamber) in N₃-conditioned medium that is from the same cell culture dish. Imaging of live neurons is performed using a TILL Photonics Imaging System with a Polychrome II monochromator and high-resolution Imago CCD camera. Cells are imaged at an exposure rate of 0.5 sec for each frame, with a total of 200 frames. For each granule, the velocity (in micrometers per second), distance (in micrometers), and direction (anterograde or retrograde) are analyzed using IP Lab software.

Oral Treatment with Fusion Protein (See, for Example, U.S. Published Application No. 2004/0013687)

Oral administrations are performed by inoculation of 1-20 micrograms of protein suspended in 100 microliters of PBS. Mice are lightly anesthetized with isoflurane (ISO-THESIA, Abbott Laboratories North, Chicago, Ill., United States of America), and protein is administered by a single application via a feeding tube. Optimized doses are given at required intervals.

Swiss-Webster mice (female, 20-25 grains each) are purchased from Ace Animals (Boyertown, Pa., U.S.A.) and allowed unrestricted access to food and water. The mice are immunized per os (p.o.). For p.o. administration, each animal is fed 4 μg of protein suspended in 0.2 mL elution buffer administered through an intragastric feeding needle. The first administration of protein occurs on day 0, and additional doses are given as optimized. Samples of serum from identically treated mice are collected and pooled 7 days after each additional dose up to three doses. For collection of serum, mice are bled with capillary tubes at the retro-orbital plexus while under isoflurane anesthesia.

Sera from treated or control mice are assayed for antibodies using immunoblot analysis. Recombinant fusion protein (0.1 μg/lane) is separated by SDS-PAGE and transferred to nitrocellulose membranes. Membranes are blocked with 5% (w/v) nonfat powdered milk in Tris-buffered saline (TBS), cut into strips and processed for detection of immunoreactive proteins using various serum samples.

Primary incubations are performed overnight (eighteen hours) at room temperature with 1:1000 diluted serum. A secondary horseradish peroxidase-labeled anti-mouse IgG is used at 1:10000 dilution for one hour at room temperature. After extensive washing, membranes are developed using enhanced chemiluminescent reagents (ECL, Amersham Biosciences, Piscataway, N.J., U.S.A.).

The toxicity of expressed proteins is tested by administering the proteins to laboratory mice. Proteins purified by elution from a histidine affinity resin or other affinity resin are diluted in PBS including 1 mg per mL bovine serum albumin (BSA) and injected intraperitoneally (i.p.) to mice. The recombinant proteins are administered in a 100 μL it aliquot of PBS-BSA at concentrations of 1 to 100 μg per animal (average weight of approximately 25 grams). Animals are monitored for varying lengths of time to detect any non-specific toxicity. Baseline physiological responses to the fusion protein are established.

### Intravenous Administration of Paclitaxel (See, for Example, Peters et al., 2007)

Experiments are performed on adult male Sprague—Dawley rats weighing 250-275 g (Harlan, Indianapolis, Ind.) at the beginning of the experiment. The rats are housed in conventional facilities with a 12 h light/dark cycle and given food and water ad libitum. All procedures are approved by the Institutional Animal Care and Use Committee at the research institution and are in accordance with National Institutes of Health guidelines for care and use of laboratory animals.

Paclitaxel is formulated by dissolving paclitaxel (Eton Bioscience, San Diego, Calif.) in a 1:1 mixture (vehicle) of ethanol and cremophor EL (CrEL) (Fluka, Denmark) to make a stock solution of 12 mg/ml. Prior to administration, the paclitaxel solution is further diluted with sterile saline (1:3) such that an intravenous (i.v.) infusion of paclitaxel dose of 18 mg/kg is delivered in a volume of 1.5 ml/250 g rat. Rats are restrained in a tail access rodent restrainer (Stoelting, Wood Dale, Ill.) and the solution is administered via tail vein over a 2 min period. A previously characterized model of PN produced by repeat infusions of paclitaxel or vehicle at a cumulative dose of 36 mg/kg (2×18 mg/kg, 3 days apart) is used as referenced in Peters et al, 2007. Previously, this dosing regimen produced a predominantly large fiber sensory neuropathy, based on electrophysiological and histological endpoints, with minimal effects on general health. Control rats received equivalent volumes of cremophor/ethanol vehicle.

### Behavioral Measurements of Paclitaxel-Induced Neuropathy

To monitor the general health of the animals, body weight is recorded and coat luster and overall appearance of the rat is noted before behavioral tests. For behavioral assays, two baseline sessions are performed on separate days prior to treatment with vehicle or paclitaxel and performances are averaged to obtain baseline values. Rats are excluded if during the two baseline sessions the rat could not consistently ambulate while on the rotarod.

Rats are behaviorally assessed 10 days post initial paclitaxel infusion. To assess changes in mechanical allodynia, von Frey microfilaments are used to determine paw withdrawal threshold. Rats are placed in a clear plastic box with a wire mesh floor and allowed to habituate for 30 min prior to testing. Von Frey microfilaments are then applied to the plantar surface of the left and right hindpaws by increasing and decreasing the stimulus intensity between 0.4 and 15.1 g equivalents of force. Each paw is tested twice with at least 10 min between trials. A positive response is noted if the paw is quickly withdrawn or licked. The 50% withdrawal threshold is found by using the “up-down” method referenced in (Peters et al., 2007). The averages of the individual paws are then averaged to find each rat's 50% withdrawal threshold.

Performance during forced ambulation is determined by assessing the rat's ability to ambulate on a rotarod apparatus (Columbus Instruments, Columbus, Ohio). Rats are placed on the rotarod for 3 min and rated on a scale of 4 to 0: (4) normal ambulation, (3) frequent paw placement errors (slips), (2) consistent paw placement errors (slips) (1), partial inability to use limbs, (0) no use of limbs. The rotarod setting was maintained at ×4 speed, 8.0 acceleration and 2.5 sensitivity. Two baseline sessions are performed on the rotarod on separate days prior to treatment of vehicle or paclitaxel and performances averaged.

Cold sensitivity/hyperalgesia is assessed by immersion of the rat's hindpaw into a water bath containing cold (4.5° C.) water, and latency to paw withdrawal was measured using a 1/100th second digital timer. Only one hindpaw is tested during each immersion, with the maximum cutoff time limited to 20 s. For each animal, left and right hindpaws are tested twice, with a minimum of 5 min between trials. The values from the two trials are averaged. The data are reported as the mean of both the right and left hindpaw values.

Sensitivity to noxious heat is measured using a Thermal Paw Stimulator (University of California, San Diego, Calif.) after a 15 min acclimation period. The intensity of radiant heat is adjusted so that the vehicle-treated rats responded to the heat by elevating or licking the hindpaw approximately 10 s after the heat is initiated. A cutoff latency of 20 s is used to prevent tissue damage. Left and right hindpaws are tested for four trials, with at least 5 min between trials. The longest latency is eliminated for each paw, and the other three trials are averaged, and values from each paw are then averaged to determine the withdrawal latency for each rat.

Using the methods above for oral administration of the fusion protein in mice, the rats are treated with fusion protein after treatment with paclitaxel and behavior is assessed as described.

### Tissue Processing and Immunohistochemistry (See, for Example, Peters et al., 2007)

Ten days following the initial paclitaxel or vehicle administration, animals are behaviorally tested, then sacrificed and processed for immunohistochemical analysis. The same procedure is used on animals treated first with taxol and subsequently treated once or more with fusion protein starting on the 10^{th} day, with an additional 10 days (minimum) of fusion protein treatment time added prior to sacrifice. Animals are perfused intracardially with 200 ml of 0.1 M phosphate buffered saline (PBS) followed by 200 ml of 4% formaldehyde/12.5% picric acid solution in 0.1 M PBS. The DRG (L3-L5), sciatic nerves, sural nerves, lumbar spinal cord and other nerves of choice are removed, post-fixed for 12 h in the perfusion fixative, and cryoprotected for 24 h in 30% sucrose in 0.1 M PBS all at 4° C. Nerves are embedded in Tissue Tek embedding media (Miles Lab, Elkhart, Ind.), rapidly frozen on dry ice, and stored at −80° C. until processed for immunohistochemistry. Longitudinal sciatic nerve sections (1.5 cm segment) are obtained at mid thigh level approximately 1.0 cm proximal to the trifurcation. Nerves are cut into 15 μm sections on a cryostat and thaw mounted on gelatin-coated slides. Spinal cord is cut into 60 μm sections on a freezing microtome and processed as free-floating sections. Sectioned tissues are incubated for 45 min at room temperature in a blocking solution of 3% normal donkey serum in PBS with 0.3% Triton-X 100 and then incubated overnight at room temperature (RT) in primary antisera against: activating transcription factor 3 (rabbit anti-ATF3, 1:500, Santa Cruz Biotechnology, Santa Cruz, Calif.), glial fibrillary acidic protein (rabbit anti-GFAP, 1:1000, Dako, Copenhagen, Denmark) or for double labeling with ATF3 (goat anti-GFAP, 1:500, Santa Cruz Biotechnology, Santa Cruz, Calif.), an antibody against CD68, a lysosomal protein present in activated macrophages (mouse anti-CD68, clone ED1, 1:5000, Serotec, Raleigh, N.C.), NF200 kD monoclonal antibody to heavy chain neurofilament (NF200, clone RT97, 1:1000, Sigma), neuronal nuclei (NeuN, 1:150, Chemicon, Temecula, Calif.), and 510013 (1;1000, Sigma, St. Louis, Mo.) to label myelinating Schwann cells. Lumbar spinal cord sections are labeled with antibodies against OX42 (CD1 1b/c, Serotec Ltd.) which labels microglia and GFAP (rabbit anti-GFAP, 1:1000, Dako, Copenhagen, Denmark) to label astrocytes. Sections are washed in PBS and incubated for 3 h at RT with secondary antibodies conjugated to various fluorescent markers (Cy2 1:200, Cy3 1:600; Jackson ImmunoResearch, West Grove, Pa.). Finally, the sections are washed 3×10 min in PBS, mounted on gelatin-coated slides, dried, dehydrated via an alcohol gradient (70, 90, and 100%), cleared in xylene, and coverslipped with DPX. To confirm the specificity of the primary antibody, controls include pre-absorption with the corresponding synthetic peptide or omission of the primary antibody. Images of immunohistochemical results are obtained using an Olympus FV1000 confocal system.

### Quantification/Image Analysis

The number of ATF3-immunoreactive (IR) cellular profiles in the nerves is counted in serial 15 μm sections from a minimum of four sections per animal. The number of cellular profiles expressing NeuN in the same nerve sections is determined to estimate the total number of neurons. The number of ATF3-IR cellular profiles is expressed as percentage of the number of NeuN-IR profiles. In order to determine the cell size distribution of ATF3 in neuronal subsets, measurements of the soma area (μm²) of approximately 1000 individual ATF3 and NeuN-IR neurons are performed using Image Pro Plus version 3.0 software (Media Cybernetics, Silver Spring, Md.). Only neurons containing a visible nucleus are counted and plotted as percentage of ATF3-IR sensory neurons within each size classification.

For analysis of immunofluorescence for GFAP in the nerves and lumbar spinal cord and for OX42 in the lumbar spinal cord, at least four randomly selected sections of the spinal cord or selected nerve is used from each animal. Images were captured on an Olympus BX51 fluorescent microscope fitted with an Olympus DP70 digital camera. The area and mean fluorescence intensity of positive staining (Integrated Optical Density, IOD) are determined within a defined fluorescence intensity threshold applied to all nerve or spinal cord sections and analyzed using Image Pro Plus software. The IOD results are expressed per total area of the given section. The IOD values for each section within an experimental animal are averaged. The IOD of nerve and spinal cord of paclitaxel-treated rats and rats treated with paclitaxel followed by fusion protein are expressed as percentage of vehicle-treated levels (100%). There are no statistically significant differences in immunofluorescence levels between vehicle-treated and age-matched naive rats.

Quantification of CD68-IR cells (activated macrophages) in sensory ganglia is determined as referenced in Peters et al., 2007. Briefly, digital grayscale images are acquired from a minimum of five ganglion sections per animal and analyzed using Image Pro Plus software. Only regions of the sensory ganglia containing sensory neuronal cell bodies (excluding peripheral nerve) are outlined. The number of CD68-IR cellular profiles per outlined area from all sections is averaged for each animal and results are expressed as total number of CD68-IR cellular profiles per unit area (mm²).

For quantification of the number of ATF3-IR and CD68-IR cells within the selected nerve, a manual counting system is used due to the greater sampling area. Sections are initially scanned at low power (×100) to identify areas with the highest density of ATF3-IR or CD68-IR cellular profiles. A 250 μm×250 μm observation field is viewed at ×400 magnification and the number of ATF3-IR or CD68-IR cells is counted. Only cells that display visible nuclei as determined by counterstaining sections with DAPI (4′,6-diainimidino-2-phenyl-indole, dihydrochloride, 1:40,000, Molecular Probes, Oreg.) are counted. Four optic fields on individual sections are assessed in at least four sections from the same nerve per animal. The results are expressed as number of ATF3-IR or CD68-IR profiles/mm².

Statistical analysis—For behavioral experiments, ANOVA followed by Student-Newman-Keuls post hoc test is used. One-way repeated measure ANOVA followed by Student-Newman—Keuls post hoc test is used to compare the % of ATF3-IR neurons in small, medium and large neurons in nerves of paclitaxel-treated rats. For the rest of comparisons, a Student's t test or Mann-Whitney Rank Sum test (when data are not parametric) is used. Results are considered statistically significant at P<0.05. In all cases, the investigator is blind to the experimental status of each animal.

### Administration of Fusion Protein to SMA Type II Mice

Mice displaying symptoms of SMA type II (Grondard et al., 2005) are administered fusion protein as described above and observed as described below. SMA type II mice are preferred because the symptoms arise later and certain treatments are able to prevent or reduce damage (Grondard et al., 2005).

### Behavioral Testing

Assay of strength in type 2 SMA-like mice—Type 2 SMA-like mice are tested to evaluate the forelimb grip strength. All of the tests are made blind, the group assignment being unknown to the observers. Control mice as well as untrained and trained type 2 SMA-like mice between 10 and 15 d of age are timed for how long they could support their weight holding onto a metal rail suspended in midair. Each mouse is subjected to five trials with at least a 10 min rest period between tests (see, for example, Grondard et al., 2005).

Open field—The ambulatory behavior is assessed in an open-field test. The apparatus consists of a wooden box measuring 28×28×5 cm. The floor of the arena is divided into 16 equal squares of 7×7 cm. Squares adjacent to walls are referred to as periphery, and the four remaining squares are referred to as center. The mice are tested individually, and the open field is washed after each session. Each mouse is placed in a central square of the open field. It is allowed to move freely for 5 min, and data are scored manually by the experimenter. The behavioral measures recorded during these 5 min were the number of peripheral and central square crossings and the percentage of peripheral crossing (see, for example, Grondard et al., 2005).

### Tissue Analysis

Histological analysis and counting motoneurons. The mice are anesthetized with chloral hydrate (3%). The lumbar region of the spinal cord (L1-L5) is processed for paraffin embedding. Two hundred twenty-five serial cross sections (12 gm thickness) of the lumbar spinal cords are made (2700 gm total length), among which one of every five sections (45 sections examined) is processed and Niss1-stained, as referenced in (Grondard et al., 2005). The sections are analyzed at a 200× magnification in the anterior horn (either left or right) for the presence of all neurons in that region. All cells are counted within the ventral horn below an arbitrary horizontal line drawn from the central canal. Only neuronal cells showing at least one nucleolus located within the nucleus are counted. Cell counts are performed using ImageJ software (National Institutes of Health, Bethesda, Md.) on images captured electronically (see, for example, Grondard et al., 2005).

Immunohistochemical analysis—Spinal cord serial sections, 50 gm thick, are cut between L1 and L5 on a sliding microtome, collected in PBS, and processed as free-floating sections. Tissue sections are incubated for 30 min at room temperature in a blocking solution (4% normal donkey serum with 0.3% Triton X-100 in PBS) and then incubated overnight at room temperature in the primary antiserum.

Immunostaining using choline acetyltransferase (ChAT) (polyclonal rabbit anti-ChAT; 1:400; Chemicon, Temecula, Calif.) is used to stain motoneurons in the spinal cord sections. After incubation, tissue sections are washed three times for 10 min in PBS and incubated in the secondary antibody solution (Alexa Fluor 488 donkey anti-rabbit IgG; 1:400; Molecular Probes, Eugene, Oreg.) for 2 h at room temperature.

Immunohistohistochemical detection of SMN protein is performed using a monoclonal antibody raised against full-length human SMN protein (1:200; clone 2B1; ImmuQuest, Cleveland, UK) and the purified rabbit polyclonal antibody H2, referenced in Grondard et al., 2005 (1:200). Sections are washed between every subsequent step with PBS. Endogenous peroxidase activity is blocked by incubating the sections in 3% H₂O₂ (diluted in PBS) for 30 min. Sections are subsequently incubated for 30 min with a biotinylated fragment of goat anti-rabbit and goat anti-mouse Ig (1:400; DakoCytomation, High Wycombe, UK), followed by horseradish peroxidase-conjugated streptavidin (DakoCytomation) and developed with DAB (DakoCytomation) chromogen to the specifications of the manufacturer.

Finally, the sections are washed three times for 10 min in PBS and mounted in Vectashield mounting medium (Vector Laboratories, Burlingame, Calif.). The staining specificity is checked in control incubations performed in the absence of the primary antibody. Motoneuron counts and areas are evaluated using ImageJ software.

Retrograde labeling of motoneurons projecting in soleus and plantaris muscles. Ten-day-old mice are anesthetized with isoflurane (Laboratoire Mundipharma, Boulogne Billancourt, France). A small incision is made in the left calf skin to expose the soleus and plantaris muscles. A total volume of 50 nl of fluorogold (Fluorochrome, Denver, Colo.) in PBS is injected in three different parts of each muscle (median, proximal, and distal) using an oil-based microinjector (Nanoject; Drummond Scientific, Broomall, Pa.). The skin is thereafter sutured with a 6-0 poly-amide thread (Supramid; S. Jackson, Alexandria, Va.), and the mice are kept at 35° C. until recovery from narcosis. They are then returned to their cage, in which all animals are given food and water ad libitum. At 13 d of age, mice arc perfused and processed for histological analysis.

Apoptosis evaluation. The apoptotic nuclei are observed after terminal deoxynucleotidyl transferase-mediated biotinylated UTP nick end-labeling (TUNEL) staining. Segments (L1-L5) embedded in paraffin are serially sectioned at 12 gm thickness. After deparaffinization and rehydration, the sections are digested for 30 min at 37° C. in proteinase K (20 μg/ml). Positive control sections from control animals are incubated in DNase 1 (1 U/10 1) for 10 min at 37° C. Tissue sections arc then processed for TUNEL staining with an in situ cell death detection kit (Roche Diagnostics, Mannheim, Germany) according to the directions of the manufacturer. Fluorescein-dUTP is used to label DNA strand breaks. For nuclear staining, sections are mounted in Vectashield mounting medium with 4′,6-diamidino-2-phenylindole (DAPI) (final concentration, 1.5 g/ml) after TUNEL staining. TUNEL-positive cells are counted at a 400× magnification on 20 sections (spanning a total interval of 2700 in) of the lumbar spinal cord of each mouse. These counts are then compared with the total number of nuclei determined after DAPI staining.

Semiquantitative and real-time RT-PCR assays. RNA is isolated using the Qiagen (Valencia, Calif.) RNeasy Mini kit according to the instructions of the manufacturer. RNA is treated with 1 U of amplification-grade deoxyribonuclease I (Invitrogen, San Diego, Calif.) per microgram of RNA to remove genomic DNA, according to the instructions of the manufacturer. Then, 0.5 g of the RNA is reverse-transcribed using Superscript 11 reverse transcriptase (Invitrogen) and treated with RNase H, according to the instructions of the manufacturer. cDNA thus obtained is then used as a template for the PCR in a 50 reaction volume including a 0.25 M concentration of each primer, 100 M dNTPs, Taq buffer, and 1 μL of Taq polymerase (ATGC Biotechnologies, Noisy-le-Grand, France). The PCR conditions for analysis of expression of each gene are designed to avoid PCR saturation and to enable semiquantitative determination. Each data point is normalized by the abundance of glyceraldehyde-3-phosphate dehydrogenase (GAPDH) mRNA. For Southern blot analysis, 15 μL of the products of each PCR are loaded on a 1% agarose gel and after electrophoresis transferred onto a Hybond-N nylon membrane (Amersham Biosciences, Arlington Heights, Ill.) and hybridized overnight at 45° C. with ³²P-labeled 20-mer primers. The primers are 32P-labeled at their 3′ ends by incorporation of [³²P]dCTP using terminal transferase (Invitrogen), according to the recommendations of the manufacturer. The blots are washed twice at room temperature with buffer containing 2×SSC and 0.1% SDS. Signals are detected by autoradiography. All of the RT-PCR experiments are repeated five times under the same conditions, and, for each gene expression analysis, the PCR is repeated twice with comparable results.

Real-time RT-PCR is performed using an ABI Prism 7700 (Applied Biosystems), and fluorescence detection is performed in 384-well plates using SYBR Green buffer (Applied Biosystems). Primer concentrations are optimized to yield the lowest concentration of primers giving the same cycle threshold (Ct) values as recommended by Applied Biosystems. A control RNA sample that was not reverse-transcribed is used with each real-time RT-PCR experiment to verify that there is no genomic DNA contamination. PCR amplification is performed (in triplicate) as a singleplex reaction in a total reaction volume of 25 μL. The reaction mixture consists of 12.5 μL of SYBR Green template (Applied Biosystems) forward and reverse primers as determined from the previous optimization procedure, nuclease-free water and cDNA. The PCR parameters are incubation for one cycle at 50° C. for 2 min to prevent amplification of carryover DNA, followed by denaturation at 94° C. for 10 min and then amplification for 40 cycles of 95° C./15 s and 60° C./1 min. Amplification products are routinely checked using dissociation curve soft ware (Applied Biosystems) and by gel electrophoresis on a 1% agarose gel and are then visualized under UV light after staining with 0.05% ethidium bromide to confirm the size of the DNA fragment and that only one product was formed. Samples are compared using the relative Ct method, where the amount of target normalized to the amount of endogenous control and relative to the control sample is given by 2AACt.

Muscle-fiber cross-sectional analysis. Frozen soleus and plantaris muscles from mice are collected and sectioned into 10-μm-thick sections. Muscle sections are stained with hematoxylin and eosin, dehydrated via an alcohol gradient (70, 90, and 100%), and mounted with Eukitt (VWR International, Strasbourg, France). The highest number of myofibers per muscle section is retained for statistical analysis.

Statistical analysis. Statistical comparisons are one-way ANOVA followed by Student's t test. For RT-PCR analysis, all values are presented as mean±SEM. Survival analysis is performed by Kaplan-Meier analysis. All data are expressed as mean±SEM. For statistical evaluation of motoneuron number, identified by either Niss1 staining or ChAT immunoreactivity, the number of cells present in each ventral horn of the L1-L5 spinal cord is counted and corrected according to the method of Abercrombie (1946), which compensates for double counting in adjacent sections.

The gene accession number for the mouse SMN is NM_{—}011420 and the gene accession number for botulinum neurotoxin B is P10844. The accession numbers for human SMN1 include AAH115308 and BC015308.

| Mouse SMN gene sequence(NM_011420) (SEQ ID NO: 5): | |
|---|---|
| 1 | gtcattgagt gagcccggca gcgtccgtgg tagcaggcca tggcgatggg cagtggcgga |
| 61 | gcgggctccg agcaggaaga tacggtgctg ttccggcgtg gcaccggcca gagtgatgat |
| 121 | tctgacattt gggatgatac agcattgata aaagcttatg ataaagctgt ggcttccttt |
| 181 | aagcatgctc taaagaacgg tgacatttgt gaaactccag ataagccaaa aggcacagcc |
| 241 | agaagaaaac ctgccaagaa gaataaaagc caaaagaaga atgccacaac tcccttgaaa |
| 301 | cagtggaaag ttggtgacaa gtgttctgct gtttggtcag aagacggctg catttaccca |
| 361 | gctactatta cgtccattga ctttaagaga gaaacctgtg tcgtggttta tactggatat |
| 421 | ggaaacagag aggagcaaaa cttatctgac ctactttccc cgacctgtga agtagctaat |
| 481 | agtacagaac agaacactca ggagaatgaa agtcaagttt ccacagacga cagtgaacac |
| 541 | tcctccagat cgctcagaag taaagcacac agcaagtcca aagctgctcc gtggacctca |
| 601 | tttcttcctc caccaccccc aatgccaggg tcaggattag gaccaggaaa gccaggtcta |
| 661 | aaattcaacg gcccgccgcc gccgcctcca ctaccccctc cccccttcct gccgtgctgg |
| 721 | atgcccccgt tcccttcagg accaccaata atcccgccac cccctcccat ctctcccgac |
| 781 | tgtctggatg acactgatgc cctgggcagt atgctaatct cttggtacat gagtggctac |
| 841 | cacactggct actatatggg tttcagacaa aataaaaaag aaggaaagtg ctcacataca |
| 901 | aattaagaag ttcagctctg tctcaggaga tggggtgtcg gtgtccctgg tcgacaagaa |
| 961 | cagacgtctc ctcgtcatca gtggactctt ggctaagtgg tgtcgtcatc agcatctccc |
| 1021 | cgctgtggga gtccatccat cctaagtcag cagcagagcg tgcctggggc gtgagcagtt |
| 1081 | ggagggaccg accagtggag tgtgcgtgtc ggaaggcagt ctacccagtc gtgactgagc |
| 1141 | acaaatgtgc aattgtcatt ttcttagcat gtcaagattt ttattaatgc ctttagaatt |
| 1201 | aaataaaagt ccttttttga aatcttg |

| Mouse SMN protein sequence (Accession No. CAA73356) (SEQ ID NO: 2): | |
|---|---|
| mamgsggags eqedtvlfrr gtgqsddsdi wddtalikay dkavasfkha lkngdicetp | 60 |
| dkpkdtarrk pakknksqkk nattplkqwk vgdkcsavws edgciypati tsidfkretc | 120 |
| vvvytgygnr eeqnlsdlls ptcevanste qntqenesqv stddsehssr slrskahsks | 180 |
| kaapwtsflp ppppmpgsgl gpgkpglkfn gpppppplpp ppflpcwmpp fpsgppiipp | 240 |
| pppispdcld dtdalgsmli swymsgyhtg yymgfrqnkk egkcshtn | 288 |
| Botulinum neurotoxin B heavy chain sequence (SEQ ID NO: 4): | |
| cidvdnedlf fiadknsfsd dlskneriey ntqsnyiend fpinelildt dliskielps | 60 |
| entesltdfn vdvpvyekqp aikkiftden tifqylysqt fpldirdisl tssfddalif | 120 |
| snkvysffsm dyiktankvv eaglfagwvk qivndfviea nksntmdkia dislivpyig | 180 |
| lalnvgneta kgnfenafei agasillefi pellipvvga fllesyidnk nkiiktidna | 240 |
| ltkrnekwsd myglivagwl stvntqfyti kegmykalny qaqaleeiik yryniyseke | 300 |
| ksninidfnd insklnegin qaidninnfi ngcsysylmk kmiplavekl ldfdntlkkn | 360 |
| llnyidenkl yligsaeyek skvnkyikti mpfdlsiytn dtiliemfnk ynseilnnii | 420 |
| lnlrykdnnl idlsgygakv evydgvelnd knqfkltssa nskirvtqnq niifnsvfld | 480 |
| fsvsfwirip kykndgiqny ihneytiinc mknnsgwkis irgnriiwtl idingktksv | 540 |
| ffeyniredi seyinrwffv titnnlnnak iyingklesn tdikdirevi angeiifkld | 600 |
| gdidrtqfiw mkyfsifnte lsqsnieery kiqsyseylk dfwgnplmyn keyymfnagn | 660 |
| knsyiklkkd spvgeiltrs kynqnskyin yrdlyigekf iirrksnsqs inddivrked | 720 |
| yiyldffnln qewrvytyky fkkeeeklfl apisdsdefy ntiqikeyde qptyscqllf | 780 |
| kkdeestdei gligihrfye sgivfeeykd yfciskwylk evkrkpynik lgcnwqfipk | 840 |
| degwte | 846 |

| Human SMN1 gene sequence (BC015308) (SEQ ID NO: 8): | |
|---|---|
| 1 | ggccccacgc tgcgcacccg cgggtttgct atggcgatga gcagcggcgg cagtggtggc |
| 61 | ggcgtcccgg agcaggagga ttccgtgctg ttccggcgcg gcacaggcca gagcgatgat |
| 121 | tctgacattt gggatgatac agcattgata aaagcatatg ataaagctgt ggcttcattt |
| 181 | aagcatgctc taaagaatgg tgacatttgt gaaacttcgg gtaaaccaaa aaccacacct |
| 241 | aaaagaaaac ctgctaagaa gaataaaagc caaaagaaga atactgcagc ttccttacaa |
| 301 | cagtggaaag ttggggacaa atgttctgcc atttggtcag aagacggttg catttaccca |
| 361 | gctaccattg cttcaattga ttttaagaga gaaacctgtg ttgtggttta cactggatat |
| 421 | ggaaatagag aggagcaaaa tctgtccgat ctactttccc caatctgtga agtagctaat |
| 481 | aatatagaac agaatgctca agagaatgaa aatgaaagcc aagtttcaac agatgaaagt |
| 541 | gagaactcca ggtctcctgg aaataaatca gataacatca agcccaaatc tgctccatgg |
| 601 | aactcttttc tccctccacc accccccatg ccagggccaa gactgggacc aggaaagcca |
| 661 | ggtctaaaat tcaatggccc accaccgcca ccgccaccac caccacccca cttactatca |
| 721 | tgctggctgc ctccatttcc ttctggacca ccaataattc ccccaccacc tcccatatgt |
| 781 | ccagattctc ttgatgatgc tgatgctttg ggaagtatgt taatttcatg gtacatgagt |
| 841 | ggctatcata ctggctatta tatgggtttt agacaaaatc aaaaagaagg aaggtgctca |
| 901 | cattccttaa attaaggaga aatgctggca tagagcagca ctaaatgaca ccactaaaga |
| 961 | aacgatcaga cagatctgga atgtgaagcg ttatagaaga taactggcct catttcttca |
| 1021 | aaatatcaag tgttgggaaa gaaaaaagga agtggaatgg gtaactcttc ttgattaaaa |
| 1081 | gttatgtaat aaccaaatgc aatgtgaaat attttactgg actctatttt gaaaaaccat |
| 1141 | ctgtaaaaga ctgaggtggg ggtgggaggc cagcacggtg gtgaggcagt tgagaaaatt |
| 1201 | tgaatgtgga ttagattttg aatgatattg gataattatt ggtaatttta tgagctgtga |
| 1261 | gaagggtgtt gtagtttata aaagactgtc ttaatttgca tacttaagca tttaggaatg |
| 1321 | aagtgttaga gtgtcttaaa atgtttcaaa tggtttaaca aaatgtatgt gaggcgtatg |
| 1381 | tggcaaaatg ttacagaatc taactggtgg acatggctgt tcattgtact gtttttttct |
| 1441 | atcttctata tgtttaaaag tatataataa aaatatttaa ttttttttta aaaaaaaaaa |
| 1501 | aaaaaaaaaa aaaaaaaaaa aaaaa |

| SEQ ID NO: 1: | |
|---|---|
| MAMSSGGSGG GVPEQEDSVL FRRGTGQSDD SDIWDDTALI KAYDKAVASF KHALKNGDIC | 60 |
| ETSGKPETTP KRKPAKKNKS QKKNTAASLQ QWKVGDKCSA IWSEDGCIYP ATIASIDFKR | 120 |
| ETCVVVYTGY GNREEQNLSD LLSPICEVAN NIEQNAQENE NESQVSTDES ENSRSPGNKS | 180 |
| DNIKPKSAPW NSFLPPPPPM PGPRLGPGKP GLKFNGPPPP PPPPPPHLLS CWLPPFPSGP | 240 |
| PIIPPPPPIC PDSLDDADAL GSMLISWYMS GYHTGYYMGF RQNQKEGRCS HSLN | 294 |
| SEQ ID NO: 3: | |
| CIKVNNWDLF FSPSEDNFTN DLDKVEEITA DTNIEAAEEN ISLDLIQQYY LTFDFDNEPE | 60 |
| NISIENLSSD IIGQLEPMPN IERFPNGKKY ELDKYTMFHY LRAQEFEHGD SRIILTNSAE | 120 |
| EALLKPNVAY TFFSSKYVKK INKAVEAFMF LNWAEELVYD FTDETNEVTT MDKIADITII | 180 |
| VPYIGPALNI GNMLSKGEFV FAIIFTGVVA MLEFIPEYAL PVFGTFAIVS YIANKVLTVQ | 240 |
| TINNALSKRN EKWDEVYKYT VTNWLAKVNT QIDLIREKMK KALENQAEAT KAIINYQYNQ | 300 |
| YTEEEKNNIN FNIDDLSSKL NESINSAMIN INKFLDQCSV SYLMNSMIDY AVKRLFDFDA | 360 |
| SVRDVLLKYI YDNRGTLVLQ VDRLKDEVNN TLSADIPFQL SKYVDNKKLL STFTEYIKNI | 420 |
| VNTSILSIVY KKDDLIDLSR YGAKINIGDR VYYDSIDKNQ IKLINLESST IEVILKNAIV | 480 |
| YNSMYENFST SFWIKIPKYF SKINLNNEYT IINCIENNSG WKVSLNYGEI IWTLQDNKQN | 540 |
| IQRVVFKYSQ MVNISDYINR WIFVTITNNR LTKSKIYING RLIDQKPISN LGNIHASNKI | 600 |
| MFKLDGCRDP RRYIMIKYFN LEDKELNEKE IKDLYDSQSN SGILKDFWGN YLQYDKPYYM | 660 |
| LNIFDPNKYV DVNNIGIRGY MYLKGPRGSV VTTNIYLNST LYEGTKFIIK KYASGNEDNI | 720 |
| VRNNDRVYIN VVVKNKEYRL ATNASQAGVE KILSALEIPD VGNLSQVVVM KSKDDQGIRN | 780 |
| KCKMNLQDNN GNDIGFIGFH LYDNIAKLVA SNWYNRQVGK ASRTFGCSWE FIPVDDGWGE | 840 |
| SSL | 843 |
| SEQ ID NO: 6: | |
| MPVTINNFNY NDPIDNNNII MMEPPFARGT GRYYKAFKIT DRIWIIPERY TFGYKPEDFN | 60 |
| KSSGIFNRDV CEYYDPDYLN TNDKKNIFLQ TMIKLFNRIK SKPLGEKLLE MIINGIPYLG | 120 |
| DRRVPLEEFN TNIASVTVNK LISNPGEVER KKGIFANLII FGPGPVLNEN ETIDIGIQNH | 180 |
| FASREGFGGI MQMKFCPEYV SVFNNVQENK GASIFNRRGY FSDPALILMH ELIHVLHGLY | 240 |
| GIKVDDLPIV PNEKKFFMQS TDAIQAEELY TFGGQDPSII TPSTDKSIYD KVLQNFRGIV | 300 |
| DRLNKVLVCI SDPNININIY KNKFKDKYKF VEDSEGKYSI DVESFDKLYK SLMFGFTETN | 360 |
| IAENYKIKTR ASYFSDSLPP VKIKNLLDNE IYTIEEGFNI SDKDMEKEYR GQNKAINKQA | 420 |
| YEEISKEHLA VYKIQMCKSV KAPGICIDVD NEDLFFIADK NSFSDDLSKN ERIEYNTQSN | 480 |
| YIENDFPINE LILDTDLISK IELPSENTES LTDFNVDVPV YEKQPAIKKI FTDENTIFQY | 540 |
| LYSQTFPLDI RDISLTSSFD DALLFSNKVY SFFSMDYIKT ANKVVEAGLF AGWVKQIVND | 600 |
| FVIEANKSNT MDKIADISLI VPYIGLALNV GNETAKGNFE NAFEIAGASI LLEFIPELLI | 660 |
| PVVGAFLLES YIDNKNKIIK TIDNALTKRN EKWSDMYGLI VAQWLSTVNT QFYTIKEGMY | 720 |
| KALNYQAQAL EEIIKYRYNI YSEKEKSNIN IDFNDINSKL NEGINQAIDN INNFINGCSV | 780 |
| SYLMKKMTPL AVEKLLDFDN TLKKNLLNYI DENKLYLIGS AEYEKSKVNK YLKTIMPFDL | 840 |
| SIYTNDTILI EMFNKYNSEI LNNIILNLRY KDNNLIDLSG YGAKVEVYDG VELNDKNQFK | 900 |
| LTSSANSKIR VTQNQNIIFN SVFLDFSVSF WIRIPKYKND GIQNYIHNEY TIINCMKNNS | 960 |
| GWKISIRGNR IIWTLIDING KTKSVFFEYN IREDISEYIN RWFFVTITNN LNNAKIYING | 1020 |
| KLESNTDIKD IREVIANGEI IFKLDGDIDR TQFIWMKYFS IFNTELSQSN IEERYKIQSY | 1080 |
| SEYLKDFWGN PLMYNKEYYM FNAGNKNSYI KLKKDSPVGE ILTRSKYNQN SKYINYRDLY | 1140 |
| IGEKFIIRRK SNSQSINDDI VRKEDYIYLD FFNLNQEWRV YTYKYFKKEE EKLFLAPISD | 1200 |
| SDEFYNTIQI KEYDEQPTYS CQLLFKKDEE STDEIGLIGI HRFYESGIVF EEYKDYFCIS | 1260 |
| KWYLKEVKRK PYNLKLGCNW QFIPKDEGWT E | 1291 |
| SEQ ID NO: 7: | |
| SLTDLGGELC IKIKNEDLTF IAEKNSFSEE PFQDEIVSYN TKNKPLNFNY SLDKITVDYN | 60 |
| LQSKITLPND RTTPVTKGIP YAPEYKSNAA STIEIHNIDD NTIYQYLYAQ KSPTTLQRIT | 120 |
| MTNSVDDALI NSTKIYSYFP SVISKVNQGA QGILFLQWVR DIIDDFTNES SQKTTIDKIS | 180 |
| DVSTIVPYIG PALNIVKQGY EGNFIGALET TGVVLLLEYI PEITLPVIAA LSIAESSTQK | 240 |
| EKIIKTIDNF LEKRYEKWIE VYKLVKAKWL GTVNTQFQKR SYQMYRSLEY QVDAIKKIID | 300 |
| YEYKIYSGPD KEQIADEINN LKNKLEEKAN KAMININIFM RESSRSFLVN QMINEAKKQL | 360 |
| LEFDTQSKNI LMQYIKANSK FIGITELKKL ESKINKVFST PIPFSYSKNL DCWVDNEEDI | 420 |
| DVILKKSTIL NLDINNDIIS DISGFNSSVI TYPDAQLVPG INGKAIHLVN NESSEVIVHK | 480 |
| AMDIEYNDMF NNFTVSFWLR VPKVSASHLE QYGTNEYSII SSMKKHSLSI GSGWSVSLKG | 540 |
| NNLIWTLKDS AGEVRQITFR DLPDKFNAYL ANKWVFITIT NDRLSSANLY INGVLMGSAE | 600 |
| ITGLGAIRED NNITLKLDRC NNNNQYVSID KFRIFCKALN PKEIEKLYTS YLSITFLRDF | 660 |
| WGNPLRYDTE YYLIPVASSS KDVQLKNITD YMYLTNAPSY TNGKLNIYYR RLYNGLKFII | 720 |
| KRYTPNNEID SFVKSGDFIK LYVSYNNNEH IVGYPKDGNA FNNLDRILRV GYNAPGIPLY | 780 |
| KKMEAVKLRD LKTYSVQLKL YDDKNASLGL VGTHNGQIGN DPNRDILIAS NWYFNHLKDK | 840 |
| ILGCDWYFVP TDEGWTND | 858 |
| SEQ ID NO: 13: | |
| MAMSSGGSGG GVPEQEDSVL FRRGTGQSDD SDIWDDTALI KAYDKAVASF KHALKNGDIC | 60 |
| ETSGKPKTTP KRKPAKKNKS QKKNTAASLQ QWKVGDKCSA IWSEDGCIYP ATIASIDFKR | 120 |
| ETCVVVYTGY GNREEQNLSD LLSPICEVAN NIEQNAQENE NESQVSTDES ENSRSPGNKS | 180 |
| DNIKPKSAPW NSFLPPPPPM PGPRLGPGKP GLKFNGPPPP PPPPPPHLLS CWLPPFPSGP | 240 |
| PIIPPPPPIC PDSLDDADAL GSMLISWYMS GYHTGYYMGF RQNQKEGRCS HSLNCIDVDN | 300 |
| EDLFFIADKN SFSDDLSKNE RIEYNTQSNY IENDFPINEL ILDTDLISKI ELPSENTESL | 360 |
| TDFNVDVPVY EKOPAINNIF TDENTIFQYL YSQTFPLDIR DISLTSSFDD ALLFSNKVYS | 420 |
| FFSMDYIKTA NKVVEAGLFA GWVKQIVNDF VIEANKSNTM DKIADISLIV PYIGLALNVG | 480 |
| NETAKGNEEN AFEIAGASIL LEFIPELLIP VVGAFLLESY IDNKNKIIKT IDNALTKRNE | 540 |
| KWSDMYGLIV AQWLSTVNTQ FYTIKEGMYK ALNYQAQALE EIIKYRYNIY SEKEKSNINT | 600 |
| DFNDINSKLN EGINQAIDNI NNFINGCSVS YLMKKMIPLA VEKLLDFDNT LKKNLLNYID | 660 |
| ENKLYLIGSA EYEKSKVNKY LKTIMPFDLS IYTNDTILIE MFNKYNSEIL NNTILNLRYK | 720 |
| DNNLIDLSGY GAKVEVYDGV ELNDKNQFKL TSSANSKIRV TQNQNIIFNS VFLDFSVSFW | 780 |
| IRIPKYKNDG IQNYIHNEYT IINCMKNNSG WKISIRGNRI IWTLIDINGN TNSVFFEYNI | 840 |
| REDISEYINR WFFVTITNNL NNAKIYINGN LESNTDIKDI REVIANGEII FKLDGDIDRT | 900 |
| QFIWMKYFSI FNTELSQSNI EERYKIQSYS EYLKDSWGNP LMYNKEYYMF NAGNKNSYIK | 960 |
| LKKDSPVGEI LTRSKYNQNS KYINYRDLYI GEKFIIRRKS NSQSINDDIV RKEDYIYLDF | 1020 |
| FNLNQEWRVY TYKYFKKEEE KLFLAPISDS DEFYNTIQIK EYDEQPTYSC QLLFKKDEES | 1080 |
| TDEIGLIGIH RFYESGIVFE EYKDYECISK WYLKEVKRKP YNLKLGCNWQ FIPKDEGWTE | 1140 |
| SEQ ID NO: 14: | |
| MAMSSGGSGG GVPEQEDSVL FRRGTGQSDD SDIWDDTALI KAYDKAVASF KHALKNGDIC | 60 |
| ETSGKPKTTP KRKPAKKNKS QKKNTAASLQ QWKVGDKCSA IWSEDGCIYP ATIASIDFKR | 120 |
| ETCVVVYTGY GNREEQNLSD LLSPICEVAN NIEQNAQENE NESQVSTDES ENSRSPGNKS | 180 |
| DNIKPKSAPW NSFLPPPPPM PGPRLGPGKP GLKFNGPPPP PPPPPPHLLS CWLPPFPSGP | 240 |
| PIIPPPPPIC PDSLDDADAL GSMLISWYMS GYHTGYYMGF RQNQKEGRCS HSLNKSVKAP | 300 |
| GICIDVDNED LFFIADKNSF SDDLSKNERI EYNTQSNYIE NDFPINELIL DTDLISKIEL | 360 |
| PSENTESLTD FNVDVPVYEK QPAIKKIFTD ENTIFQYLYS QTFPLDIRDI SLTSSFDDAL | 420 |
| LFSNKVYSFF SMDYINTANK VVEAGLFAGW VNQIVNDFVI EANKSNTMDK IADISLIVPY | 480 |
| IGLALNVGNE TAKGNFENAF EIAGASILLE FIPELLIPVV GAFLLESYID NKNKIIKTID | 540 |
| NALTKRNEKW SDMYGLIVAQ WLSTVNTQFY TIKEGMYKAL NYQAQALEEI IKYRYNIYSE | 600 |
| KEKSNINIDF NDINSKLNEG INQAIDNINN FINGCSVSYL MKKMIPLAVE KLLDFDNTLK | 660 |
| KNLLNYIDEN KLYLIGSAEY EKSKVNKYLK TIMPFDLSIY TNDTILIEMF NKYNSEILNN | 720 |
| IILNLRYKDN NLIDLSGYGA KVEVYDGVEL NDKNQFKLTS SANSKIRVTQ NQNIIENSVF | 780 |
| LDFSVSFWIR IPKYKNDGIQ NYIHNEYTII NCMKNNSGWK ISIRGNRIIW TLIDINGKTK | 840 |
| SVFFEYNIRE DISEYINRWF FVTITNNLNN AKIYINGKLE SNTDIKDIRE VIANGEIIFK | 900 |
| LDGDIDRTQF IWMKYFSIFN TELSQSNIEE RYKIQSYSEY LKDFWGNPLM YNKEYYMFNA | 960 |
| GNKNSYIKLK KDSPVGEILT RSKYNQNSKY INYRDLYIGE KFIIRRKSNS QSINDDIVRK | 1020 |
| EDYIYLDFFN LNQEWRVYTY KYFKKEEEKL FLAPISDSDE FYNTIQIKEY DEQPTYSCQL | 1080 |
| LFKKDEESTD EIGLIGIHRE YESGIVFEEY KDYFCISKWY LKEVKRKPYN LKLGCNWQFI | 1140 |
| PKDEGWTE | 1148 |
| SEQ ID NO: 15: | |
| MAMSSGGSGG GVPEQEDSVL FRRGTGQSDD SDIWDDTALI KAYDKAVASE KHALKNGDIC | 60 |
| ETSGKPKTTP KRKPAKKNKS QKKNTAASLQ QWKVGDKCSA IWSEDGCIYP ATIASIDFKR | 120 |
| ETCVVVYTGY GNREEQNLSD LLSPICEVAN NIEQNAQENE NESQVSTDES ENSRSPGNKS | 180 |
| DNIKPKSAPW NSFLPPPPPM PGPRLGPGKP GLKFNGPPPP PPPPPPHLLS CWLPPFPSGP | 240 |
| PIIPPPPPIC PDSLDDADAL GSMLISWYMS GYHTGYYMGF RQNQKEGRCS HSLNKKAPGI | 300 |
| CIDVDNEDLF FIADKNSFSD DLSKNERIEY NTQSNYIEND FPINELILDT DLISKIELPS | 360 |
| ENTESLTDFN VDVPVYEKQP AIKKIFTDEN TIFQYLYSQT FPLDIRDISL TSSFDDALLF | 420 |
| SNKVYSFFSM DYIKTANKVV EAGLFAGWVK QIVNDFVIEA NKSNTMDKIA DISLIVPYIG | 480 |
| LALNVGNETA KGNFENAFEI AGASILLEFI PELLIPVVGA FLLESYIDNK NKIIKTIDNA | 540 |
| LTKRNEKWSD MYGLIVAQWL STVNTQFYTI KEGMYKALNY QAQALEEIIK YRYNIYSEKE | 600 |
| KSNINIDFND INSKLNEGIN QAIDNINNFI NGCSVSYLMK KMIPLAVFKL LDFDNTLKKN | 660 |
| LLNYIDENKL YLIGSAEYEK SKVNKYLKTI MPFDLSIYTN DTILTEMFNK YNSEILNNII | 720 |
| LNLRYKDNNL IDLSGYGAKV EVYDGVELND KNQFKLTSSA NSKIRVTQNQ NIIFNSVFLD | 780 |
| FSVSFWIRIP KYKNDGIQNY IHNEYTIINC MKNNSGWKIS IRGNRIIWTL IDINGKTKSV | 840 |
| FFEYNIREDI SEYINRWFFV TITNNLNNAK IYINGKLESN TDIKDIREVI ANGEIIFKLD | 900 |
| GDIDRTQFIW MKYFSIENTE LSQSNIEERY KIQSYSEYLK DFWGNPLMYN KEYYMFNAGN | 960 |
| KNSYIKLKKD SPVGEILTRS KYNQNSKYIN YRDLYIGEKF IIRRKSNSQS INDDIVRKED | 1020 |
| YIYLDFFNLN QEWRVYTYKY FKKEEEKLFL APISDSDEFY NTIQIKEYDE QPTYSCQLLF | 1080 |
| KKDEESTDEI GLIGIHRFYE SGIVFFEYKD YFCISKWYLK EVKRKPYNLK LGCNWQFIPK | 1140 |
| DEGWTE | 1146 |

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

U.S. Pat. No. 7,368,532
U.S. Pat. No. 6,159,931
U.S. Pat. No. 5,034,322
U.S. Pat. No. 5,106,739
U.S. Pat. No. 5,625,136
U.S. Pat. No. 4,938,949
U.S. Published Application No. 2005/0143289
U.S. Published Application No. 2004/0013687
Abercrombie, M., 1946 “Estimation of nuclear population from microtome sections” Anat Rec, 94:239-247
Altschul, S. F. et al. 1990 “Basic Local Alignment Search Tool” J. Mol. Biol. 215:403-410.
Altschul, S. F. et al. 1997 “Gapped BLAST and PSI-BLAST: A New Generation of Protein Database Search Programs” Nucl. Acids Res. 25:3389-3402.
Argyriou et al., 2008, Peripheral nerve damage associated with administration of taxanes in patients with cancer. Crit. Rev Oncol Hematol, 66(3): p. 218-28
Balabanian, S., N. H. Gendron, and A. E. MacKenzie, 2007, Histologic and transcriptional assessment of a mild SMA model. Neurol Res, 29(5): p. 413-24
Baldwin, M. R., J. J. Kim, and J. T. Barbieri, 2007, Botulinum neurotoxin B-host receptor recognition: it takes two receptors to tango. Nat Struct Mol Biol, 14(1): p. 9-10
Beltz, G. A., Jacobs, K. A., Eickbush, T. H., Cherbas, P. T., Kafatos, F. C., 1983 “Isolation of multigene families and determination of homologies by filter hybridization methods” Methods of Enzymology, R. Wu, L. Grossman and K. Moldave [eds.] Academic Press, New York 100:266-285.
Burnett, B. G. Eric Munoz, Animesh Tandon, Deborah Y. Kwon, Charlotte J. Sumner, and Kenneth H. Fischbeck “Regulation of SMN Protein Stability” Molecular and Cellular Biology, March 2009, 29(5):1107-1115
Carrel, T. L. et al., 2006, Survival motor neuron function in motor axons is independent of functions required for small nuclear ribonucleoprotein biogenesis. J Neurosci, 26(43): p. 11014-22
Chai, Q. et al., 2006, Structural basis of cell surface receptor recognition by botulinum neurotoxin B. Nature, 444(7122): p. 1096-1100
Cifuentes-Diaz, C. et al., 2002, Neurofilament accumulation at the motor endplate and lack of axonal sprouting in a spinal muscular atrophy mouse model. Hum Mol Genet, 11(12): p. 1439-47
de Boer, H. A., Comstock, L. J., Vasser, M. (1983) “The tac promoter: a functional hybrid derived from the trp and lac promoters” Proc. Natl. Acad. Sci. USA 80(1):21-25
Fazio et al., 1999, Docetaxel neuropathy: a distal axonopathy. Acta Neuropathol, 98(6): p. 651-3
Francis et al., 2004, A survival motor neuron:tetanus toxin fragment C fusion protein for the targeted delivery of SMN protein to neurons. Brain Res, 995(1): p. 84-96
Ganasia-Leymarie et al., 2003, Signal transduction pathways of taxanes-induced apoptosis. Curr Med Chem Anticancer Agents, 3(4): p. 291-306
Gong et al., 2002, Localization of major gangliosides in the PNS: implications for immune neuropathies. Brain, 125(Pt 11): p. 2491-506
Goode, G. B. and Shearn, D. L., 1982, Botulism: a case with associated sensory abnormalities. Arch Neurol, 39(1): p. 55
Grondard et al., 2005, Regular exercise prolongs survival in a type 2 spinal muscular atrophy model mouse. J Neurosci, 25(33): p. 7615-22
Gunawardena, S, and Goldstein, L. S., 2004, Cargo-carrying motor vehicles on the neuronal highway: transport pathways and neurodegenerative disease. J Neurobiol, 58(2): p. 258-71
Horwitz, S. B. 1994, Taxol (paclitaxel): mechanisms of action. Ann Oncol, 5 Suppl 6: p. S3-6
Hua et al., 2008, Antisense masking of an hnRNP A1/A2 intronic splicing silencer corrects SMN2 splicing in transgenic mice. Am J Hum Genet, 82(4): p. 834-48
Jablonka et al., 2006, Distinct and overlapping alterations in motor and sensory neurons in a mouse model of spinal muscular atrophy. Hum Mol Genet, 15(3): p. 511-8
Jimenez-Andrade, 2006, Sensory neurons and their supporting cells located in the trigeminal, thoracic and lumbar ganglia differentially express markers of injury following intravenous administration of paclitaxel in the rat. Neurosci Lett, 405(1-2): p. 62-7
Karlin, S, and Altschul, S. F. 1990 “Methods for Assessing the Statistical Significance of Molecular Sequence Features by Using General Scoring Schemes” Proc. Natl. Acad. Sci. USA 87:2264-2268.
Karlin, S, and Altschul, S. F. 1993 “Applications and Statistics for Multiple High-Scoring Segments in Molecular Sequences” Proc. Natl. Acad. Sci. USA 90:5873-5877.
Kuruoglu, R., B. Cengiz, and A. Tokcaer, 1996, Botulism with sensory symptoms diagnosed by neuromuscular transmission studies associated with edrophonium responsiveness. Electromyogr Clin Neurophysiol, 36(8): p. 477-80
Lalli et al., 2003, The journey of tetanus and botulinum neurotoxins in neurons. Trends Microbiol, 11(9): p. 431-7
Lee, J. J. and Swain, S. M., 2006, Peripheral neuropathy induced by microtubule-stabilizing agents. J Clin Oncol, 24(10): p. 1633-42
Li, J. Y., R. Jahn, and A. Dahlstrom. 1994, Synaptotagmin I is present mainly in autonomic and sensory neurons of the rat peripheral nervous system. Neuroscience, 63(3): p. 837-50
Maniatis, T., Fritsch, E. F., Sambrook, J. 1982, Molecular Cloning: A Laboratory Manual, Cold Spring Harbor Laboratory, Cold Spring Harbor, N.Y.
Markman, M., 2003, Managing taxane toxicities. Support Care Cancer, 11(3): p. 144-7
Markowitz, J. A., M. B. Tinkle, and K. H. Fischbeck, 2004, Spinal muscular atrophy in the neonate. J Obstet Gynecol Neonatal Nurs, 33(1): p. 12-20
Martinez-Castrillo et al., 1991, Botulism with sensory symptoms: a second case. J Neurol Neurosurg Psychiatry, 54(9): p. 844-5
McWhorter et al., 2003, Knockdown of the survival motor neuron (Smn) protein in zebrafish causes defects in motor axon outgrowth and pathfinding. J Cell Biol, 162(5): p. 919-31
Meng et al., 2007, Synaptobrevin I mediates exocytosis of CGRP from sensory neurons and inhibition by botulinum toxins reflects their anti-nociceptive potential. J Cell Sci, 120(Pt 16): p. 2864-74
Mielke, S., A. Sparreboom, and K. Mross, 2006, Peripheral neuropathy: a persisting challenge in paclitaxel-based regimes. Eur J Cancer, 42(1): p. 24-30
Monani et al., 2003, A transgene carrying an A2G missense mutation in the SMN gene modulates phenotypic severity in mice with severe (type I) spinal muscular atrophy. J Cell Biol, 160(1): p. 41-52
Omran et al., 1998, Axonal neuropathy and predominance of type II myofibers in infantile spinal muscular atrophy. J Child Neurol, 13(7): p. 327-31
Pellizzoni, L. et al., 2002, Purification of native survival of motor neurons complexes and identification of Gemin6 as a novel component. J Biol Chem, 277(9): p. 7540-5
Peters et al., 2007, Intravenous paclitaxel administration in the rat induces a peripheral sensory neuropathy characterized by macrophage infiltration and injury to sensory neurons and their supporting cells. Exp Neurol, 203(1): p. 42-54
Rao, M. V. and Nixon, R. A., 2003, Defective neurofilament transport in mouse models of amyotrophic lateral sclerosis: a review. Neurochem Res, 28(7): p. 1041-7
Rudnik-Schoneborn et al., 2003, Classical infantile spinal muscular atrophy with SMN deficiency causes sensory neuronopathy. Neurology, 60(6): p. 983-7
Rummel et al., 2007, Identification of the protein receptor binding site of botulinum neurotoxins B and G proves the double-receptor concept. Proc Natl Acad Sci USA, 104(1): p. 359-64
Sahenk et al., 1994, Taxol neuropathy. Electrodiagnostic and sural nerve biopsy findings. Arch Neurol, 51(7): p. 726-9
Schmalbruch, H. and Haase, G. 2001, Spinal muscular atrophy: present state. Brain Pathol, 11(2): p. 231-47
Shanmugarajan et al., 2007, Congenital bone fractures in spinal muscular atrophy: functional role for SMN protein in bone remodeling. J Child Neurol, 22(8): p. 967-73
Simpson, L. L., A. B. Maksymowych, and N. Kiyatkin, 1999, Botulinum toxin as a carrier for oral vaccines. Cell Mol Life Sci, 56(1-2): p. 47-61
Sonnabend et al., 1987, Intestinal toxicoinfection by Clostridium botulinum type F in an adult. Case associated with Guillain-Barre syndrome. Lancet, 1(8529): p. 357-61
Terns. M. P. and Terns, R. M., 2001, Macromolecular complexes: SMN—the master assembler. Curr Biol, 11(21): p. R862-4
Viollet et al., 1997, cDNA isolation, expression, and chromosomal localization of the mouse survival motor neuron gene (Smn). Genomics, 40(1): p. 185-8
Vitte et al., 2004, Deletion of murine Smn exon 7 directed to liver leads to severe defect of liver development associated with iron overload. Am J Pathol, 165(5): p. 1731-41
Vyas et al., 2002, Involvement of survival motor neuron (SMN) protein in cell death. Hum Mol Genet, 11(22): p. 2751-64
Xu, D., McElroy, D., Thornburg, R. W., Wu, R. et al. (1993) “Systemic induction of a potato pin2 promoter by wounding, methyl jasmonate, and abscisic acid in transgenic rice plants” Plant Molecular Biology 22:573-588
Zhai, R. G. and Bellen, H. J., 2004, Hauling t-SNAREs on the microtubule highway. Nat Cell Biol, 6(10): p. 918-9
Zhang et al., 2003, Active transport of the survival motor neuron protein and the role of exon-7 in cytoplasmic localization. J Neurosci, 23(16): p. 6627-37
Zhang et al., 2008, SMN deficiency causes tissue-specific perturbations in the repertoire of snRNAs and widespread defects in splicing. Cell, 133(4): p. 585-600
Zhou, Y. and Singh, B. R., 2004, Cloning, high-level expression, single-step purification, and binding activity of His6-tagged recombinant type B botulinum neurotoxin heavy chain transmembrane and binding domain. Protein Expr Purif, 34(1): p. 8-16
Zou et al., 2007, SMN protects cells against mutant SOD1 toxicity by increasing chaperone activity. Biochem Biophys Res Commun, 364(4): p. 850-5

1. A fusion protein comprising a survival motor neuron protein (SMN), or a fragment thereof having SMN biological activity, and a non-toxic botulinum neurotoxin (BoNT), or a fragment thereof capable of providing for receptor-mediated endocytosis into a cell.

2. The fusion protein according to claim 1, wherein said non-toxic BoTN optionally comprises a modified or hybrid polypeptide, wherein said modified or hybrid polypeptide comprises an amino acid sequence or polypeptide from a non-BoTN protein or polypeptide.

3. The fusion protein according to claim 1, wherein said non-toxic BoTN comprises a cell binding domain or moiety and/or a cell membrane translocation domain or moiety.

4. The fusion protein according to claim 2, wherein said modified and/or hybrid polypeptide comprises a cell binding domain or moiety and/or a cell membrane translocation domain or moiety.

5. The fusion protein according to claim 2, wherein said modified or hybrid polypeptide comprises a non-toxic portion of a diphtheria toxin and/or a tetanus toxin.

6. The fusion protein according to claim 5, wherein said non-toxic portion of diphtheria toxin comprises a cell membrane translocation domain.

7. The fusion protein according to claim 5, wherein said tetanus toxin comprises the heavy chain of tetanus toxin, or a fragment thereof capable of providing for cell binding and/or membrane translocation.

8. The fusion protein according to claim 7, wherein said heavy chain of tetanus toxin comprises SEQ ID NO:7, or a fragment thereof capable of providing for cell binding and/or membrane translocation.

9. The fusion protein according to claim 5, wherein said tetanus toxin is modified to reduce or eliminate immunogenic epitopes.

10. The fusion protein according to claim 1, wherein said BoTN comprises the BoTN heavy chain.

11. The fusion protein according to claim 10, wherein said BoTN heavy chain comprises a cell binding or recognition domain and/or a membrane translocation domain optionally modified to reduce or eliminate immunogenic epitopes and/or polypeptide aggregation.

12. The fusion protein according to claim 1, wherein said BoTN is serotype A or B.

13. The fusion protein according to claim 1, wherein said BoTN comprises the amino acid sequence of SEQ ID NO:3 or SEQ ID NO:4, or a fragment thereof capable of providing for receptor-mediated endocytosis in a cell.

14. The fusion protein according to claim 1, wherein said SMN protein is a mammalian SMN protein.

15. The fusion protein according to claim 1, wherein said SMN protein is a human SMN1 protein.

16. The fusion protein according to claim 1, wherein said SMN protein comprises the amino acid sequence shown in SEQ ID NO:1, or a fragment thereof having SMN biological activity.

17. The fusion protein according to claim 1, wherein the survival motor neuron protein and the botulinum neurotoxin heavy chain are connected through an interchain amino acid segment or linker; or by a chemical moiety linking group; or by way of a disulfide bond.

18-19. (canceled)

20. The fusion protein according to claim 1, wherein the survival motor neuron protein and the botulinum neurotoxin heavy chain are connected via both an interchain amino acid segment or linker and a disulfide bond between cysteine amino acids in the survival motor neuron protein and botulinum neurotoxin heavy chain portions.

21. The fusion protein according to claim 1, wherein said fusion protein comprises the amino acid sequence shown in any of SEQ ID NO:13, SEQ ID NO:14, or SEQ ID NO:15, or a fragment thereof having SMN biological activity and capable of providing for receptor-mediated endocytosis in a cell.

22-26. (canceled)

27. A method for treating, inhibiting the progression of, or preventing a disorder associated with and/or characterized by neuronal degeneration in a person or animal, said method comprising administering to the person or animal an effective amount of a fusion protein comprising a survival motor neuron protein (SMN), or a fragment thereof having SMN biological activity, and a non-toxic botulinum neurotoxin (BoNT), or a fragment thereof capable of providing for receptor-mediated endocytosis into a cell; or a composition comprising said fusion protein; or a polynucleotide encoding said fusion protein.

28-36. (canceled)

37. A method for transporting survival motor neuron protein to axon terminals of a neuron by way of receptor-mediated endocytosis, said method comprising contacting the neuron with a fusion protein comprising a survival motor neuron protein (SMN), or a fragment thereof having SMN biological activity, and a non-toxic botulinum neurotoxin (BoNT), or a fragment thereof capable of providing for receptor-mediated endocytosis into a cell or a composition comprising said fusion protein; or a polynucleotide encoding said fusion protein.

38-41. (canceled)


SEQUENCE LISTING (17 sequences)

SEQ ID NO: 1 - protein, 294 aa, Homo sapiens
MAMSSGGSGGGVPEQEDSVLFRRGTGQSDDSDIWDDTALIKAYDKAVASFKHALKNGDICETSGKPKTTPKRKPAKKNKSQKKNTAASLQQWKVGDKCSAIWSEDGCIYPATIASIDFKRETCVVVYTGYGNREEQNLSDLLSPICEVANNIEQNAQENENESQVSTDESENSRSPGNKSDNIKPKSAPWNSFLPPPPPMPGPRLGPGKPGLKFNGPPPPPPPPPPHLLSCWLPPFPSGPPIIPPPPPICPDSLDDADALGSMLISWYMSGYHTGYYMGFRQNQKEGRCSHSLN

SEQ ID NO: 2 - protein, 288 aa, Mus musculus
MAMGSGGAGSEQEDTVLFRRGTGQSDDSDIWDDTALIKAYDKAVASFKHALKNGDICETPDKPKDTARRKPAKKNKSQKKNATTPLKQWKVGDKCSAVWSEDGCIYPATITSIDFKRETCVVVYTGYGNREEQNLSDLLSPTCEVANSTEQNTQENESQVSTDDSEHSSRSLRSKAHSKSKAAPWTSFLPPPPPMPGSGLGPGKPGLKFNGPPPPPPLPPPPFLPCWMPPFPSGPPIIPPPPPISPDCLDDTDALGSMLISWYMSGYHTGYYMGFRQNKKEGKCSHTN

SEQ ID NO: 3 - protein, 843 aa, Clostridium botulinum
CIKVNNWDLFFSPSEDNFTNDLDKVEEITADTNIEAAEENISLDLIQQYYLTFDFDNEPENISIENLSSDIIGQLEPMPNIERFPNGKKYELDKYTMFHYLRAQEFEHGDSRIILTNSAEEALLKPNVAYTFFSSKYVKKINKAVEAFMFLNWAEELVYDFTDETNEVTTMDKIADITIIVPYIGPALNIGNMLSKGEFVEAIIFTGVVAMLEFIPEYALPVFGTFAIVSYIANKVLTVQTINNALSKRNEKWDEVYKYTVTNWLAKVNTQIDLIREKMKKALENQAEATKAIINYQYNQYTEEEKNNINFNIDDLSSKLNESINSAMININKFLDQCSVSYLMNSMIPYAVKRLKDFDASVRDVLLKYIYDNRGTLVLQVDRLKDEVNNTLSADIPFQLSKYVDNKKLLSTFTEYIKNIVNTSILSIVYKKDDLIDLSRYGAKINIGDRVYYDSIDKNQIKLINLESSTIEVILKNAIVYNSMYENFSTSFWIKIPKYFSKINLNNEYTIINCIENNSGWKVSLNYGEIIWTLQDNKQNIQRVVFKYSQMVNISDYINRWIFVTITNNRLTKSKIYINGRLIDQKPISNLGNIHASNKIMFKLDGCRDPRRYIMIKYFNLFDKELNEKEIKDLYDSQSNSGILKDFWGNYLQYDKPYYMLNLFDPNKYVDVNNIGIRGYMYLKGPRGSVVTTNIYLNSTLYEGTKFIIKKYASGNEDNIVRNNDRVYINVVVKNKEYRLATNASQAGVEKILSALEIPDVGNLSQVVVMKSKDDQGIRNKCKMNLQDNNGNDIGFIGFHLYDNIAKLVASNWYNRQVGKASRTFGCSWEFIPVDDGWGESSL

SEQ ID NO: 4 - protein, 846 aa, Clostridium botulinum
CIDVDNEDLFFIADKNSFSDDLSKNERIEYNTQSNYIENDFPINELILDTDLISKIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIVNDFVIEANKSNTMDKIADISLIVPYIGLALNVGNETAKGNFENAFEIAGASILLEFIPELLIPVVGAFLLESYIDNKNKIIKTIDNALTKRNEKWSDMYGLIVAQWLSTVNTQFYTIKEGMYKALNYQAQALEEIIKYRYNIYSEKEKSNINIDFNDINSKLNEGINQAIDNINNFINGCSVSYLMKKMIPLAVEKLLDFDNTLKKNLLNYIDENKLYLIGSAEYEKSKVNKYLKTIMPFDLSIYTNDTILIEMFNKYNSEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEEKLFLAPISDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 5 - DNA, 1227 bp, Mus musculus
gtcattgagtgagcccggcagcgtccgtggtagcaggccatggcgatgggcagtggcggagcgggctccgagcaggaagatacggtgctgttccggcgtggcaccggccagagtgatgattctgacatttgggatgatacagcattgataaaagcttatgataaagctgtggcttcctttaagcatgctctaaagaacggtgacatttgtgaaactccagataagccaaaaggcacagccagaagaaaacctgccaagaagaataaaagccaaaagaagaatgccacaactcccttgaaacagtggaaagttggtgacaagtgttctgctgtttggtcagaagacggctgcatttacccagctactattacgtccattgactttaagagagaaacctgtgtcgtggtttatactggatatggaaacagagaggagcaaaacttatctgacctactttccccgacctgtgaagtagctaatagtacagaacagaacactcaggagaatgaaagtcaagtttccacagacgacagtgaacactcctccagatcgctcagaagtaaagcacacagcaagtccaaagctgctccgtggacctcatttcttcctccaccacccccaatgccagggtcaggattaggaccaggaaagccaggtctaaaattcaacggcccgccgccgccgcctccactaccccctccccccttcctgccgtgctggatgcccccgttcccttcaggaccaccaataatcccgccaccccctcccatctctcccgactgtctggatgacactgatgccctgggcagtatgctaatctcttggtacatgagtggctaccacactggctactatatgggtttcagacaaaataaaaaagaaggaaagtgctcacatacaaattaagaagttcagctctgtctcaggagatggggtgtcggtgtccctggtcgacaagaacagacgtctcctcgtcatcagtggactcttggctaagtggtgtcgtcatcagcatctccccgctgtgggagtccatccatcctaagtcagcagcagagcgtgcctggggcgtgagcagttggagggaccgaccagtggagtgtgcgtgtcggaaggcagtctacccagtcgtgactgagcacaaatgtgcaattgtcattttcttagcatgtcaagatttttattaatgcctttagaattaaataaaagtccttttttgaaatcttg

SEQ ID NO: 6 - protein, 1291 aa, Clostridium botulinum
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSVKAPGICIDVDNEDLFFIADKNSFSDDLSKNERIEYNTQSNYIENDFPINELILDTDLISKIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIVNDFVIEANKSNTMDKIADISLIVPYIGLALNVGNETAKGNFENAFEIAGASILLEFIPELLIPVVGAFLLESYIDNKNKIIKTIDNALTKRNEKWSDMYGLIVAQWLSTVNTQFYTIKEGMYKALNYQAQALEEIIKYRYNIYSEKEKSNINIDFNDINSKLNEGINQAIDNINNFINGCSVSYLMKKMIPLAVEKLLDFDNTLKKNLLNYIDENKLYLIGSAEYEKSKVNKYLKTIMPFDLSIYTNDTILIEMFNKYNSEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEEKLFLAPISDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 7 - protein, 858 aa, Clostridium tetani
SLTDLGGELCIKIKNEDLTFIAEKNSFSEEPFQDEIVSYNTKNKPLNFNYSLDKIIVDYNLQSKITLPNDRTTPVTKGIPYAPEYKSNAASTIEIHNIDDNTIYQYLYAQKSPTTLQRITMTNSVDDALINSTKIYSYFPSVISKVNQGAQGILFLQWVRDIIDDFTNESSQKTTIDKISDVSTIVPYIGPALNIVKQGYEGNFIGALETTGVVLLLEYIPEITLPVIAALSIAESSTQKEKIIKTIDNFLEKRYEKWIEVYKLVKAKWLGTVNTQFQKRSYQMYRSLEYQVDAIKKIIDYEYKIYSGPDKEQIADEINNLKNKLEEKANKAMININIFMRESSRSFLVNQMINEAKKQLLEFDTQSKNILMQYIKANSKFIGITELKKLESKINKVFSTPIPFSYSKNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTND

SEQ ID NO: 8 - DNA, 1525 bp, Homo sapiens
ggccccacgctgcgcacccgcgggtttgctatggcgatgagcagcggcggcagtggtggcggcgtcccggagcaggaggattccgtgctgttccggcgcggcacaggccagagcgatgattctgacatttgggatgatacagcactgataaaagcatatgataaagctgtggcttcatttaagcatgctctaaagaatggtgacatttgtgaaacttcgggtaaaccaaaaaccacacctaaaagaaaacctgctaagaagaataaaagccaaaagaagaatactgcagcttccttacaacagtggaaagttggggacaaatgttctgccatttggtcagaagacggttgcatttacccagctaccattgcttcaattgattttaagagagaaacctgtgttgtggtttacactggatatggaaatagagaggagcaaaatctgtccgatctactttccccaatctgtgaagtagctaataatatagaacagaatgctcaagagaatgaaaatgaaagccaagtttcaacagatgaaagtgagaactccaggtctcctggaaataaatcagataacatcaagcccaaatctgctccatggaactcttttctccctccaccaccccccatgccagggccaagactgggaccaggaaagccaggtctaaaattcaatggcccaccaccgccaccgccaccaccaccaccccacttactatcatgctggctgcctccatttccttctggaccaccaataattcccccaccacctcccatatgtccagattctcttgatgatgctgatgctttgggaagtatgttaatttcatggtacatgagtggctatcatactggctattatatgggttttagacaaaatcaaaaagaaggaaggtgctcacattccttaaattaaggagaaatgctggcatagagcagcactaaatgacaccactaaagaaacgatcagacagatctggaatgtgaagcgttatagaagataactggcctcatttcttcaaaatatcaagtgttgggaaagaaaaaaggaagtggaatgggtaactcttcttgattaaaagttatgtaataaccaaatgcaatgtgaaatattttactggactctattttgaaaaaccatctgtaaaagactgaggtgggggtgggaggccagcacggtggtgaggcagttgagaaaatttgaatgtggattagattttgaatgatattggataattattggtaattttatgagctgtgagaagggtgttgtagtttataaaagactgtcttaatttgcatacttaagcatttaggaatgaagtgttagagtgtcttaaaatgtttcaaatggtttaacaaaatgtatgtgaggcgtatgtggcaaaatgttacagaatctaactggtggacatggctgttcattgtactgtttttttctatcttctatatgtttaaaagtatataataaaaatatttaatttttttttaaaaaaaaaaaaaaaaaaaaaaaaaaaaaaaaaaaa

SEQ ID NO: 9 - protein, 25 aa, Artificial Sequence
  amino acids 300-324 of Ewings sarcoma protein (Sigma)
RGRGRGGFDRGGMSRGGRGGGRGGM

SEQ ID NO: 10 - protein, 8 aa, Artificial Sequence
  fusion protein
DYKDDDDK

SEQ ID NO: 11 - protein, 8 aa, Artificial Sequence
  interchain amino acid segment
KSVKAPGI

SEQ ID NO: 12 - protein, 6 aa, Artificial Sequence
  interchain amino acid segment
KKAPGI

SEQ ID NO: 13 - protein, 1140 aa, Artificial Sequence
  fusion protein
MAMSSGGSGGGVPEQEDSVLFRRGTGQSDDSDIWDDTALIKAYDKAVASFKHALKNGDICETSGKPKTTPKRKPAKKNKSQKKNTAASLQQWKVGDKCSAIWSEDGCIYPATIASIDFKRETCVVVYTGYGNREEQNLSDLLSPICEVANNIEQNAQENENESQVSTDESENSRSPGNKSDNIKPKSAPWNSFLPPPPPMPGPRLGPGKPGLKFNGPPPPPPPPPPHLLSCWLPPFPSGPPIIPPPPPICPDSLDDADALGSMLISWYMSGYHTGYYMGFRQNQKEGRCSHSLNCIDVDNEDLFFIADKNSFSDDLSKNERIEYNTQSNYIENDFPINELILDTDLISKIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIVNDFVIEANKSNTMDKIADISLIVPYIGLALNVGNETAKGNFENAFEIAGASILLEFIPELLIPVVGAFLLESYIDNKNKIIKTIDNALTKRNEKWSDMYGLIVAQWLSTVNTQFYTIKEGMYKALNYQAQALEEIIKYRYNIYSEKEKSNINIDFNDINSKLNEGINQAIDNINNFINGCSVSYLMKKMIPLAVEKLLDFDNTLKKNLLNYIDENKLYLIGSAEYEKSKVNKYLKTIMPFDLSIYTNDTILIEMFNKYNSEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEEKLFLAPISDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 14 - protein, 1148 aa, Artificial Sequence [sequence omitted]

SEQ ID NO: 15 - protein, 1146 aa, Artificial Sequence
  fusion protein
MAMSSGGSGGGVPEQEDSVLFRRGTGQSDDSDIWDDTALIKAYDKAVASFKHALKNGDICETSGKPKTTPKRKPAKKNKSQKKNTAASLQQWKVGDKCSAIWSEDGCIYPATIASIDFKRETCVVVYTGYGNREEQNLSDLLSPICEVANNIEQNAQENENESQVSTDESENSRSPGNKSDNIKPKSAPWNSFLPPPPPMPGPRLGPGKPGLKFNGPPPPPPPPPPHLLSCWLPPFPSGPPIIPPPPPICPDSLDDADALGSMLISWYMSGYHTGYYMGFRQNQKEGRCSHSLNKKAPGICIDVDNEDLFFIADKNSFSDDLSKNERIEYNTQSNYIENDFPINELILDTDLISKIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIVNDFVIEANKSNTMDKIADISLIVPYIGLALNVGNETAKGNFENAFEIAGASILLEFIPELLIPVVGAFLLESYIDNKNKIIKTIDNALTKRNEKWSDMYGLIVAQWLSTVNTQFYTIKEGMYKALNYQAQALEEIIKYRYNIYSEKEKSNINIDFNDINSKLNEGINQAIDNINNFINGCSVSYLMKKMIPLAVEKLLDFDNTLKKNLLNYIDENKLYLIGSAEYEKSKVNKYLKTIMPFDLSIYTNDTILIEMFNKYNSEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEEKLFLAPISDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 16 - protein, 10 aa, Artificial Sequence
  interchain amino acid segment
CGLVPAGSGP

SEQ ID NO: 17 - protein, 17 aa, Artificial Sequence
  interchain amino acid segment
CGLVPAGSGPSAGSSAC